(19) United States
(12) Patent Application Publication
(10) Pub. No.: US 2010/0168535 A1
(43) Pub. Date: Jul. 1, 2010

(54) METHODS AND APPARATUSES RELATED TO BLOOD ANALYTE MEASUREMENT SYSTEM

(76) Inventors: Mark Ries Robinson, Albuquerque, NM (US); William R. Patterson, Irvine, CA (US); Richard P. Thompson, Dana Point, CA (US); Shonn Hendee, Carlsbad, CA (US); James H. Macemon, Poway, CA (US)

Correspondence Address: The Grafe Law Office, P.C., P.O. Box 2689, Corrales, NM, 87048, US

(21) Appl. No.: 12/714,100

(22) Filed: Feb. 26, 2010

Related U.S. Application Data
(63) Continuation-in-part of application No. 11/679,835, filed on Feb. 27, 2007.
(60) Provisional application No. 60/791,719, filed on Apr. 12, 2006.

(51) Int. Cl. A61B 5/00

(52) U.S. Cl. 600/309

(57) ABSTRACT

The present invention relates to a blood analyte measurement system for the procurement of blood samples for measurement of blood properties such as analyte concentration or analyte presence. A blood access system can be coupled with a measurement system such as an electrochemical sensor, and can also be used with other measurement modalities. Embodiments of the present invention can facilitate accurate measurement of blood glucose by the clinician in a sterile manner. Embodiments of the present invention can also enable the calibration of the sensor at one or more calibration points. One desired analyte of measurement is glucose for the effective implementation of glycemic control protocols. Embodiments of the present invention can also be used for the measurement of other analytes such as arterial blood gases, lactate, hemoglobin, potassium and urea. Additionally, embodiments of the present invention can function effectively on a variety of blood access points and specifically enables glucose monitoring in an existing arterial line that is already in place for hemodynamic monitoring. The present invention does not consume a significant amount of blood. Some embodiments of the present invention can re-infuse the blood into the patient, which can facilitate operation of the system in a sterile manner.

### CROSS REFERENCE TO RELATED APPLICATIONS

This application claims priority to U.S. provisional 60/791,719 filed Apr. 12, 2006, and claims priority as a continuation-in-part of Ser. No. 11/679,835 filed Feb. 27, 2007, which claimed priority to U.S. provisional 60/791,719 filed Apr. 12, 2006, each of which is incorporated herein by reference. This application is related to the following patent applications, each of which is incorporated herein by reference: Ser. No. 11/679,839, filed Feb. 28, 2007; Ser. No. 11/679,837, filed Feb. 28, 2007; Ser. No. 11/679,826, filed Feb. 27, 2007; and PCT/US2006/060850, filed Nov. 13, 2006.

### BACKGROUND

More than 20 peer-reviewed publications have demonstrated that control of blood glucose significantly improves critical care patient outcomes. Glycemic control (GC) has been shown to reduce surgical site infections by 60% in cardiothoracic surgery patients and reduce overall ICU mortality by 40% with significant reductions in ICU morbidity and length of stay. See, e.g., Furnary Tony, Oral presentation at 2005 ADA annual, session titled “Management of the Hospitalized Hyperglycemic Patient;” Van den Berghe et al., NEJM 2001; 345:1359. Historically, caregivers have treated hyperglycemia (high blood glucose) only when glucose levels exceeded 220 mg/dl. Based upon recent clinical findings, however, experts now recommend IV insulin administration to control blood glucose to within the normoglycemic range (80-110 mg/dl). Adherence to such strict glucose control regimens requires near-continuous monitoring of blood glucose and frequent adjustment of insulin infusion to achieve normoglycemia while avoiding risk of hypoglycemia (low blood glucose). In response to the demonstrated clinical benefit, approximately 90% of US hospitals have adopted some form of glycemic control protocol and are using it on approximately 80% of patients, regardless of diabetes status.

Given the evidence for improved clinical outcomes associated with glycemic control, hospitals are under pressure to implement GC as the standard of practice for critical care and cardiac surgery patients. Clinicians and caregivers have developed GC protocols that use IV insulin administration to maintain more normal patient glucose levels. To be safe and effective, these protocols require frequent blood glucose monitoring. Currently, these protocols involve periodic removal of blood samples by nursing staff and testing on handheld meters or blood gas analyzers. Although hospitals are responding to the identified clinical need, adoption has been difficult with current technology due to two principal reasons.

Fear of hypoglycemia. The target glucose range of 80-110 mg/dl brings the patient near clinical hypoglycemia (blood glucose less than 50 mg/dl). Patients exposed to hypoglycemia for greater than 30 minutes have significant risk of neurological damage. IV insulin administration with only intermittent glucose monitoring (typically hourly by most GC protocols) exposes patients to increased risk of hypoglycemia. In a recent letter to the editors of Intensive Care medicine, it was noted that 42% of patients treated with a GC protocol in the UK experienced at least one episode of hypoglycemia. See, e.g., lain Mackenzie et al., “Tight glycaemic control: a survey of intensive care practice in large English Hospitals;” Intensive Care Med (2005) 31:1136. In addition, handheld meters require procedural steps that are often cited as a source of measurement error, further exacerbating the fear (and risk) of accidentally taking the blood glucose level too low. See, e.g., Bedside Glucose Testing systems, CAP today, April 2005, page 44.

Burdensome procedure. Most glycemic control protocols require frequent glucose monitoring and insulin adjustment at 30 minute to 2 hour intervals (typically hourly) to achieve normoglycemia. Caregivers recognize that glucose control would be improved with continuous or near-continuous monitoring. Unfortunately, existing glucose monitoring technology is incompatible with the need to obtain frequent measurements. Using current technology, each measurement requires removal of a blood sample, performance of the blood glucose test, evaluation of the result, determination of the correct therapeutic action, and finally adjustment to the insulin infusion rate. High measurement frequency requirements coupled with a labor-intensive and time-consuming test places significant strain on limited ICU nursing resources that already struggle to meet patient care needs.

There is a need for improved methods and apparatuses of measuring analytes in patients, especially for measuring analytes such as glucose without requiring burdensome nurse interaction, significant blood loss, or increasing risk of infection.

### SUMMARY OF THE INVENTION

The present invention relates to a blood analyte measurement system for the procurement of blood samples for measurement of blood properties such as analyte concentration or analyte presence. A blood access system can be coupled with a measurement system such as an electrochemical sensor, and can also be used with other measurement modalities. Embodiments of the present invention can facilitate accurate measurement of blood glucose by the clinician in a sterile manner. Embodiments of the present invention can also enable the calibration of the sensor at one or more calibration points. One desired analyte of measurement is glucose for the effective implementation of glycemic control protocols. Embodiments of the present invention can also be used for the measurement of other analytes such as arterial blood gases, lactate, hemoglobin, potassium and urea. Additionally, embodiments of the present invention can function effectively on a variety of blood access points and specifically enables glucose monitoring in an existing arterial line that is already in place for hemodynamic monitoring. The present invention does not consume a significant amount of blood. Some embodiments of the present invention can re-infuse the blood into the patient, which can facilitate operation of the system in a sterile manner.

Some embodiments of the present invention provide an apparatus for measuring an analyte in blood taken from a patient, comprising (a) a patient interface device, capable of interfacing with the circulatory system of a patient; (b) an analyte sensor having first and second ports, with the first port in fluid communication with the patient interface device; (c) a flow generation and reservoir system having first and second ports, with the first port in fluid communication with second port of the analyte sensor; and (d) a first fluid source, mounted such that it can be placed in fluid communication with the second port of the flow generation and storage system, wherein the first fluid source provides a first fluid having a first predetermined analyte concentration. Some embodiments further comprise a flow divider in fluid communication with the first port of the analyte sensor and with the second port of the analyte sensor. Some embodiments further comprise a pressure monitor in fluid communication with the circulatory system of a patient.

Some embodiments further comprise a second fluid source mounted such that it can be placed in fluid communication with the second port of the flow generation and reservoir system, wherein the second fluid source provides a second fluid having a second predetermined analyte concentration, different from the first analyte concentration. Some such embodiments further comprise a first fluid selection system in fluid communication with the first and second fluid sources and with the second port of the flow generation and reservoir system, wherein the first fluid selection system has first and second configurations, wherein in the first configuration only the first fluid is supplied to the flow generation and reservoir system, and in the second configuration only the second fluid is supplied to the flow generation and reservoir system.

Some embodiments further comprise a plurality of additional fluid sources mounted such that each additional fluid source can be placed in fluid communication with the second port of the flow generation and reservoir system, wherein each additional fluid source provides an additional fluid having a predetermined analyte concentration, wherein the analyte concentration of the first fluid and of the additional fluids are different from each other. Some such embodiments further comprise a fluid selection system in fluid communication with the first fluid source and with each of the additional fluid sources and with the second port of the flow generation and reservoir system, and wherein the fluid control system has a plurality of configurations, wherein for the first fluid source and for each of the additional fluid sources there is a configuration of the first fluid selection system that allows only said fluid source to supply fluid to the flow generation and reservoir system.

Some embodiments further comprise a second fluid source mounted such that it can be placed in fluid communication with the second port of the analyte sensor, wherein the second fluid source provides a second fluid having a second predetermined analyte concentration, different from the first analyte concentration. Some such embodiments further comprise a first fluid selection system in fluid communication with the second port of the analyte sensor, with the first port of the flow generation and reservoir system, and with the second fluid source, wherein the fluid selection system has a first configuration in which the second port of the analyte sensor is in fluid communication with the second port of the flow generation and reservoir system and not with the second fluid source, and a second configuration in which the first port of the flow generation and reservoir system is in fluid communication with the second fluid source and not with the second port of the analyte sensor. In some such embodiments the first fluid selection system comprises one or more multiway valves, or a plurality of shutoff valves, or a plurality of pinch clamps, or a combination thereof. Some embodiments further comprise a waste channel mounted such that it can be placed in fluid communication with the first port of the analyte sensor. Some such embodiments further comprise a second fluid selection system in fluid communication with the first port of the analyte sensor, with the patient interface device, and with the waste channel, wherein the second fluid selection system has a first configuration in which the first port of the analyte sensor is in fluid communication with the patient interface device and not with the waste channel, and a second configuration in which the first port of the analyte sensor is in fluid communication with the waste channel and not with the patient interface device.

Some embodiments further comprise a waste channel mounted such that it can be placed in fluid communication with the first port of the analyte sensor. Some such embodiments further comprise a first fluid selection system in fluid communication with the first port of the analyte sensor, with the patient interface device, and with the waste channel, wherein the first fluid selection system has a first configuration in which the first port of the analyte sensor is in fluid communication with the patient interface device and not with the waste channel, and a second configuration in which the first port of the analyte sensor is in fluid communication with the waste channel and not with the patient interface device.

Some embodiments further comprise a first access port in fluid communication with the first port of the analyte sensor and allowing fluid communication with the first port of the analyte sensor, and further comprising a second access port in fluid communication with the second port of the analyte sensor allowing fluid communication with the second port of the analyte sensor.

Some embodiments further comprise a flow divider in fluid communication with the first port of the analyte sensor and with the second port of the analyte sensor, wherein the fluid pathway from the patient interface device to the first port of the analyte sensor has a first flow cross-section, the fluid pathway through the analyte sensor has a second flow cross-section, and the fluid bypass has a third flow cross-section, wherein the first flow cross-section is larger than the third flow cross-section, and the third flow cross-section is larger than the second flow cross-section.

In some embodiments the flow generation and reservoir system comprises a syringe pump having first and second ports, the first port in fluid communication with the second port of the analyte sensor and the second port in fluid communication with the fluid selection device.

In some embodiments, the flow generation and reservoir system comprises a syringe pump. In some embodiments, the flow generation and reservoir system comprises a peristaltic pump having first and second ports, and a reservoir having first and second ports, wherein the first port of the peristaltic pump is in fluid communication with the second port of the reservoir, and wherein the first port of the reservoir is in fluid communication with the analyte sensor, and wherein the second port of the peristaltic pump is mounted such that it can be placed in fluid communication with the first fluid source. In some such embodiments, the reservoir comprises one or more of a bag, a flexible pillow, a syringe, a bellows device, a device that can be expanded through pressure, and an expandable fluid column. In some embodiments, the flow generation and reservoir system comprises a peristaltic pump having first and second ports, and a reservoir having a first port, wherein the first port of the peristaltic pump comprises the first port of the flow generation and reservoir system, and wherein the second port of the peristaltic pump is in fluid communication with the first port of the reservoir.

Some embodiments, further comprise a second fluid source, and wherein the flow generation and reservoir system comprises a first syringe pump and a second syringe pump, wherein the first syringe pump is in fluid communication with the first fluid source, and wherein the second syringe pump is in fluid communication with the second fluid source, and wherein the first syringe pump and second syringe pump are each in fluid communication with the second port of the analyte sensor. In some such embodiments, the first syringe pump is connected to the second port of the analyte sensor through a first flow interrupting device, and wherein the second syringe pump is connected to the second port of the analyte sensor through a second flow interrupting device.

Some embodiments further comprise a second fluid source, and wherein the flow generation and reservoir system comprises a first reservoir and a second reservoir and a peristaltic pump having first and second ports, wherein the first reservoir is in fluid communication with the first fluid source, and wherein the second reservoir is in fluid communication with the second fluid source, and wherein the first reservoir and second reservoirs are each in fluid communication with the second port of the peristaltic pump, and wherein the first port of the peristaltic pump is in fluid communication with the second port of the analyte sensor. In some such embodiments, the first reservoir is connected to the second port of the peristaltic pump through a first flow interrupting device, and wherein the second reservoir is connected to the second port of the peristaltic pump through a second flow interrupting device.

Some embodiments of the present invention provide a method of measuring an analyte concentration, comprising (a) providing an apparatus as described in this specification; (b) operating the flow generation and reservoir system to place the first fluid in operative contact with the analyte sensor; (c) determining a calibration responsive to the analyte sensor output while in operative contact with the first fluid; (d) operating the flow generation and storage system to place blood from the patient in operative contact with the analyte sensor; and (e) determining the analyte concentration from the analyte sensor output while in operative contact with blood and from the calibration.

Some embodiments of the present invention provide a method of measuring an analyte concentration, comprising (a) providing an apparatus as in described in this specification; (b) operating the flow generation and reservoir system to place the first fluid in operative contact with the analyte sensor; (c) operating the flow generation and storage system to place the second fluid in operative contact with the analyte sensor; (d) determining a calibration responsive to the analyte sensor output while in operative contact with the first fluid and the analyte sensor output while in operative contact with the second fluid; (e) operating the flow generation and storage system to place blood from the patient in operative contact with the analyte sensor; and (f) determining the analyte concentration from the analyte sensor output while in operative contact with blood and from the calibration.

Some embodiments of the present invention provide a method of measuring an analyte concentration, comprising (a) providing an apparatus as described in this specification; (b) operating the flow generation and reservoir system to place the first fluid in operative contact with the analyte sensor; (c) operating the flow generation and reservoir system to place the second fluid in operative contact with the analyte sensor; (d) determining a calibration responsive to the analyte sensor output while in operative contact with the first fluid and the analyte sensor output while in operative contact with the second fluid; (e) operating the flow generation and reservoir system to place blood from the patient in operative contact with the analyte sensor; and (f) determining the analyte concentration from the analyte sensor output while in operative contact with blood and from the calibration.

Some embodiments of the present invention provide an apparatus for measuring an analyte in blood taken from a patient, comprising (a) a patient interface device, capable of interfacing with the circulatory system of a patient; (b) an analyte sensor having first and second ports, with the first port in fluid communication with the patient interface device; (c) a flow generation and reservoir system having first and second ports, with the first port in fluid communication with second port of the analyte sensor; (d) a first fluid source, mounted such that it can be placed in fluid communication with the second port of the flow generation and reservoir system, wherein the first fluid source provides a first fluid having a first predetermined analyte concentration; and (e) a second fluid source, mounted such that it can be placed in fluid communication with the second port of the analyte sensor, wherein the second fluid source provides a second fluid having a second predetermined analyte concentration, where the second predetermined analyte concentration is different than the first predetermined analyte concentration.

Some embodiments of the present invention provide an apparatus for the measurement of an analyte, comprising (a) a patient interface device capable of interfacing with the circulatory system of a patient; (b) an analyte sensor having first and second ports, with the first port in fluid communication with the patient interface device; (c) a flow generation device having first and second ports, with the first port in fluid communication with second port of the analyte sensor; (d) a waste channel in fluid communication with the second port of the flow generation device through a first flow control device that allows fluid flow from the flow generation device to the waste channel but substantially prevents fluid from the waste channel to the flow generation device; and (e) a first fluid source, mounted such that it can be placed in fluid communication with the second port of the flow generation device through a second flow control device that allows fluid flow from the first fluid source to the flow generation device but substantially prevents fluid from the flow generation device to the first fluid source, wherein the first fluid source provides a first fluid having a first predetermined analyte concentration. Some such embodiments 38 further comprise a second fluid source, mounted such that it can be placed in fluid communication with the second port of the flow generation device through a third flow control device that allows fluid flow from the second fluid source to the flow generation device but substantially prevents fluid from the flow generation device to the second fluid source, wherein the second fluid source provides a first fluid having a second predetermined analyte concentration, and where the second predetermined analyte concentration is different than the first predetermined analyte concentration. Some such embodiments further comprise a first fluid selection system in fluid communication with the first and second fluid sources and with the second port of the flow generation device, wherein the first fluid selection system has first and second configurations, wherein in the first configuration only the first fluid is supplied to the flow generation device, and in the second configuration only the second fluid is supplied to the flow generation device.

Some embodiments further comprise a first fluid selection system in fluid communication with the second port of the analyte sensor, with the first port of the flow generation device, and with the second fluid source, wherein the fluid selection system has a first configuration in which the second port of the analyte sensor is in fluid communication with the second port of the flow generation device and not with the second fluid source, and a second configuration in which the first port of the flow generation device is in fluid communication with the second fluid source and not with the second port of the analyte sensor.

Some embodiments of the present invention provide a method of measuring an analyte, comprising: (a) providing an apparatus as described in this specification; (b) placing the patient interface device in fluid communication with the vascular system of a patient; (c) operating the flow generation device to place the first fluid in operative contact with the analyte sensor and determining a response of the analyte sensor responsive to the first fluid; (d) operating the flow generation device to move fluid from the analyte sensor to the waste channel and to place blood from the patient in operative contact with the analyte sensor and determining a response of the analyte sensor responsive to the blood; and (e) determining an analyte measurement from the responses of the analyte sensor.

Some embodiments of the present invention provide a method of measuring an analyte, comprising (a) providing an apparatus as described in this specification; (b) placing the patient interface device in fluid communication with the vascular system of a patient; (c) operating the flow generation device to place the first fluid in operative contact with the analyte sensor and determining a response of the analyte sensor responsive to the first fluid; (d) operating the flow generation device to place the second fluid in operative contact with the analyte sensor and determining a response of the analyte sensor responsive to the second fluid; (e) operating the flow generation device to move fluid from the analyte sensor to the waste channel and to place blood from the patient in operative contact with the analyte sensor and determining a response of the analyte sensor responsive to the blood; and (f) determining an analyte measurement from the responses of the analyte sensor.

Some embodiments of the present invention provide a method of measuring an analyte using at least two calibration fluids, wherein the analyte sensor is placed in operative contact with the second fluid after being in operative contact with the first fluid and before being in operative contact with blood, and wherein the analyte concentration of the second fluid is less than the analyte concentration of the first fluid.

### BRIEF DESCRIPTION OF THE DRAWINGS

The accompanying figures are incorporated into and form part of the specification, and, with the specification, illustrate example embodiments of the present invention.

FIG. 1 is a schematic depiction of an example embodiment of the present invention having a syringe push-pull operation.

FIG. 2 is a schematic depiction of an example embodiment of the present invention having a syringe push-pull operation with an added calibration bag.

FIG. 3 is a schematic depiction of an example embodiment of the present invention having a push-pull operation.

FIG. 4 is a schematic depiction of an example embodiment of the present invention with a sensor close to a reservoir.

FIG. 5 is a schematic depiction of an example embodiment of the present invention with a sensor close to a patient.

FIG. 6 is a schematic depiction of an example embodiment of the present invention with a calibration bypass circuit.

FIG. 7 is a schematic depiction of an example embodiment of the present invention with a waste pathway.

FIG. 8 is a schematic depiction of an example embodiment of the present invention with a calibration pathway circuit and a waste pathway circuit.

FIG. 9 is a schematic depiction of an example embodiment of the present invention with a sensor with manual access.

FIG. 10 is a schematic depiction of an example embodiment of the present invention with two syringes.

FIG. 11 is a schematic depiction of an example embodiment of the present invention with two reservoirs and a peristaltic pump.

FIG. 12 is a schematic depiction of an example embodiment of the present invention with a peristaltic pump and reservoir.

FIG. 13 is a schematic depiction of an example embodiment of the present invention with a flow divider bypass circuit.

FIG. 14 is a schematic depiction of an example embodiment of a flow divider.

FIG. 15 is a schematic depiction of an example embodiment of the present invention including a sensor bypass loop.

FIG. 16 is a schematic depiction of an example embodiment of the present invention illustrating a general system configuration.

FIG. 17 is a schematic depiction of an example embodiment of the present invention illustrating a general system configuration.

FIG. 18 shows several reaction equations and the resulting products that lead to sensor suppression.

FIG. 19 shows a blood access circuit with two potential fluid sources and enabling the use of a low concentration maintenance fluid.

FIG. 20 shows a blood access circuit with two potential fluid sources and enabling the use of a low concentration maintenance fluid.

FIG. 21 shows a blood access circuit with two potential fluid sources and enabling the use of a low concentration maintenance fluid.

### DETAILED DESCRIPTION OF THE PRESENT INVENTION

The present invention relates to a blood analyte measurement system for the procurement of blood samples for measurement of blood properties such as analyte concentration or analyte presence. A blood access system can be coupled with a measurement system such as an electrochemical sensor, and can also be used with other measurement modalities. Embodiments of the present invention can facilitate accurate measurement of blood glucose by the clinician in a sterile manner. Embodiments of the present invention can also enable the calibration of the sensor at one or more calibration points. One desired analyte of measurement is glucose for the effective implementation of glycemic control protocols. Embodiments of the present invention can also be used for the measurement of other analytes such as arterial blood gases, lactate, hemoglobin, potassium and urea. Additionally, embodiments of the present invention can function effectively on a variety of blood access points and specifically enables hemodynamic monitoring. The present invention does not consume a significant amount of blood. Some embodiments of the present invention can re-infuse the blood into the patient, which can facilitate operation of the system in a sterile manner. A blood access system suitable for the applications mentioned above can have any one or combination of several desirable characteristics, described below.

Blood loss. In many applications, the system will be used to make multiple measurements over the course of operation. Accordingly, it is desirable that the system minimize blood loss incident to each measurement. A system according to the present invention can measure the blood by an electrochemical sensor. Such a measurement method need not consume any blood.

Blood Damage. To further minimize blood loss, the blood can be withdrawn, measured, and then re-infused. The system should avoid damage to the blood and avoid activation of the blood in a manner that would harm the patient. An example of blood damage that should be avoided includes lysis or breaking of the red blood cells. Shear stress on blood is also known to create cell lysis and activation of the clotting cascade. Activation should be avoided as it increases the propensity for clotting. For example, Von Willebrand factor (vWF) is a blood glycoprotein involved in hemostasis. It is know that high shear rates will result in activation of this protein and subsequent clotting. The avoidance of high shear stress can be difficult when a system component generates significant flow restriction. For example, if the sensor has a small cross sectional area, the shear stress on both the blood and the sensor will be high. Such situations should be avoided or minimized if possible. Embodiments of the present invention provide for movement of blood into and out of the system in a manner that does not damage or activate the blood removed from the patient. One example embodiment uses a syringe although other pressure generating mechanisms can be utilized, including peristaltic pumps.

Minimization of Clot Formation. The removal of blood for the body and interaction with a foreign surface can result in clot formation. The formation of clots is typically exacerbated by creating areas of stagnation, rough surfaces, areas of turbulent flow and any device that traps platelets. For the reduction of clot formation it may be advantageous to use a variety of anti-thrombogenic coatings or to add anticoagulation to the fluid used in the system. Types of anti-thrombogenic coating can take many forms with several different types disclosed below. Some anti-thrombogenic coatings contain both releasable and covalently bonded anti-thrombogenic agents such as heparin to provide short and long-term protection against thrombus formation. Other coatings mimic the surface of endothelial cells of blood vessels. By mimicking the surface of endothelial cells, the coating prevents the adhesion of fibrinogen and platelets to the surface of the polymer, thereby preventing the initiation of the clotting cascade. Additionally, by preventing the formation of clots and thromboses, the risk of bacterial film formation is dramatically reduced. Other coatings rely on a lubricious coating that is often hydrophilic. The combination of a smooth surface and the hydrophilic nature of the coating decrease clot formation.

Anticoagulants can be added to the fluids used in the system. These fluid sources are broadly defined as any non-blood fluid used in the system and include both calibration and maintenance fluid. The use of anticoagulants helps to facilitate cleaning of the circuit, reduce protein buildup on the sensing element, reduce cellular aggregation or platelet adhesion to the circuit. As examples, heparin and citrate can each be used as additives that reduce the possibility of cellular aggregation. In the case of heparin, it can be added to a saline bag or a calibration fluid bag. Due to the calcium binding effects of citrate, citrate might be added to either the saline or calibration bag while calcium may be added to the other bag. Such a methodology can provide for anticoagulation of the blood while also providing a means for replacing any bound calcium by the direct infusion of calcium during the administration of calibration fluid to keep the access site open. One of ordinary skill in the art will recognize that a number of additional additives can be placed in the system fluids for the prevention of clot formation.

Saline Infusion. The blood access system can use fluid sources such as saline as a mechanism for cleaning the system of blood and for pushing the blood back into the patient. The amount of saline re-infused can be an important parameter as the amount of re-infused saline should not be such that the patient becomes volume overloaded. As mixing occurs between the blood and saline, systems minimizing the amount of mixing are desired.

Some example embodiments provide for minimization of mixing use low turbulent draw methods and tubing with low shear forces at the walls. Other considerations include the number of discontinuities included in the system, the number of luer connections and any discontinuity where cells can become trapped via stagnation. In some embodiments, the saline used for the final washing and subsequent cleaning of the circuit can be pumped to waste. The use of a waste or cleaning loop can provide multiple avenues for decreasing the saline infused into the patient.

Multiple Blood Access Points. Patients in the intensive care unit can have a multitude of blood access points, including peripheral venous access, arterial access, pulmonary artery catheters, central venous lines, peripherally inserted central catheter, and others that provide access to a blood sample. It can be desirable that a system have the capability of being attached to any of these access points. Therefore, the blood analyte measurement system must be able to manage or compensate for different vascular pressures. Some embodiments of the present invention enable blood pressure monitoring.

Compensation for Catheter Types. The types of catheters used for various access points can vary in characteristics such as length, volume and mixing characteristics. A system that can work with a variety of catheter types can be desirable.

Concurrent Pressure Monitoring. Pressure monitoring is commonly done in the intensive care unit and the operating room. Arterial lines are used for general systemic pressure monitoring while central venous pressure monitoring and pulmonary artery monitoring provide venous return and pulmonary vasculature pressure measurements. It can be desirable that a system according to the present invention using one of these access points provide minimal disruption to the pressure monitoring system, disrupt the pressure monitoring system in a repeatable fashion, or in some other manner compensate for the disruption. Some embodiments of the present invention enable standard pressure monitoring to occur between measurements. The pressure monitoring device can be located on a fluid pathway that is in fluid communication with the subject. In most embodiments, the pressure transducer is located close to the flow generation device but such a restriction in placement is not required. In fact the pressure monitoring device can be located on any fluid pathway that allows for accurate pressure measurements including waste pathways, calibration pathways, etc.

Access to Blood Sample. As the blood access system procures a blood sample for measurement, it can be desired that a conventional blood sample be obtained. The sample obtained may be used for other diagnostic purposes or to assess system operation. A check sample is a blood sample obtained to assess the overall operation of the blood analyte measurement system. When a check sample is used to examine performance, the ability to obtain a blood sample from the same access point at the same time can be very beneficial. Such capability is especially important when the blood analyte measurement system is attached to an arterial site. If the blood analyte measurement system does not provide a mechanism for obtaining a sample from the same access site, the clinician is forced to make an additional arterial measurement which is considered very difficult or utilize a venous reference. As glucose values between arterial sources, capillary sources and venous sources differ, the use of an unmatched reference can add significant reference error. “Previous studies have observed large differences between capillary and venous BG values collected from patients in non-fasting conditions” Capillary and Venous Blood Glucose Concentrations Measured During Intravenous Insulin and Glucose Infusion: A Comparison of Steady and Dynamic States by Kempe et al, DIABETES TECHNOLOGY & THERAPEUTICS, Volume 11, Number 10, 2009. Additional information on this difference between different blood sources can be found in Kuwa K, Nakayama T, Hoshino T, Tominaga M: Relationships of glucose concentrations in capillary whole blood, venous whole blood and venous plasma. Clin Chim Acta 2001; 307:187-192; Colagiuri S, Sandbaek A, Carstensen B, Christensen J, Glumer C, Lauritzen T, Borch-Johnsen K: Comparability of venous and capillary glucose measurements in blood. Diabet Med 2003; 20:953-956; Larsson-Cohn U: Differences between capillary and venous blood glucose during oral glucose tolerance tests. Scand J Clin Lab Invest 1976; 36:805-808; Eriksson KF, Fex G, Trell E: Capillary-venous differences in blood glucose values during the oral glucose tolerance test, Clin Chem 1983; 29:993. Smaller but significant errors can be introduced by using venous blood samples from different arms with the same type of blood access or different sources, for example a venous arm access site compared with a central venous catheter. Depending on the heat or vasodilatation of the vascular system, the amount of glucose extracted by the capillary bed can vary appreciably. The influence of vasodilatation on the extraction of glucose by the capillary bed has been documented in several articles including arterial, arterialized venous, venous and capillary blood glucose measurements in a normal man during hyperinsulinaemic euglycaemia and hypoglycaemia, Liu et al, Diabetologia (1992) 35:287-290. Based upon the differences in glucose concentration between sources and over time, the established best way to minimize such undesired reference errors is to use a check sample from the same site and at the same time.

A conventional blood sample can be obtained from the blood analyte measurement system by a standard syringe or a standard blood tube. The system may provide feedback, e.g., audio or visual signals or instructions, to aid the attendant in procuring the sample. Some embodiments fill a syringe by pumping from the system; others allow the attendant to manually withdraw a sample. The blood access point can be attached to any portion of the circuit that provides a representative sample. If air bubble introduction is a concern, the system may automatically detect the presence of any air bubbles that might be re-infused into the patient and provide warning to the operator.

Validation or Calibration Sample. In some applications, it can be desirable for the blood access system to provide the ability to introduce and subsequently measurement a validation or calibration sample. Such a sample can be placed in the access system or provided in a manner that mimics a sample in the access system. Some embodiments of the present invention provide for a solution to be injected into the blood access system, or injected directly into the sensor.

Another embodiment uses an electronic check-sample to introduce a characteristic voltage or current signal into the instrumentation that verifies the performance of subsequent electronic and computational stages. One embodiment can mimic the detector signal with repeatable voltage waveforms produced by a digital-to-analog converter. These waveforms can mimic known amounts of the glucose signal to verify calibration accuracy.

Keep Vein Open Capability. The maintenance of blood access devices can be facilitated by the use of a “keep vein open” (KVO) infusion into the vascular system. KVO infusions are used on both arterial and venous access points. In practice the vascular point can be kept open by the infusion of about 3 ml/hr of intravenous solution. Some embodiments of the present invention provide a capability to infuse solution at a similar rate to maintain movement of blood or saline across the catheter for the minimization of clot formation. This fluid infusion can be accomplished by gravity flow, a pressurized bag or other means.

Cleaning Capability. It can be desirable for a system to have a cleaning capability, or example to reduce general contamination of the blood tubing and measurement system, the formation of small clots, or for general maintenance of the system. A solution used for cleaning the system can be infused into the patient or can be emptied into a waste bag. A solution used to push blood back into the patient can also accomplish cleaning of the system. Blood can often be a difficult substance to clean from a fluid management system. Accordingly, a cleaning cycle can utilize variable rates of flow, changes in direction of flow, and vibrate modes. A vibrate mode can take many forms; for example, the operator could push on the syringe then stop and push again. Such a push-stop-push technique is commonly used to clean peripherally inserted central catheters.

Two Part Cleaning. In some applications, it can be desirable to clean portions of the system with an enhanced cleaner such as one containing a detergent, surfactant, emulsifier, soap or the like. Example additives include polyethylene glycol or carboxy methyl cellulose. The enhanced cleaner can be used throughout the measurement cycle or introduced into the circuit during the end of an infusion cycle. In some use cases, the infusion cycle can be stopped before a significant portion (e.g., any, or any amount over some threshold) of the enhanced cleaner reaches the patient. A subsequent recirculation or cleaning cycle can cause the enhanced cleaner to flow through the system (but not enter the patient). A non-enhanced cleaner (e.g., saline) can be introduced into the circuit following the enhanced cleaner, such that the enhanced cleaner flows through the system, followed by the non-enhanced cleaner. The volumes of non-enhanced cleaner and enhanced cleaner can be controlled such that enhanced cleaner is not left in a portion of the system where it can be infused into the patient. In some applications, the useful life of the system can be extended by periodic cleaning with an cleaning agent.

Disconnect Detection. The blood access system can contain a method for determining when the system becomes disconnected from the patient. For example, pressure detection, air detection, or the use of sound waves can be used to indicate that the system is not attached to a patient.

Air-in-line Detection. The blood access system can detect and prevent the infusion of air bubbles into the vascular system in any of several ways. For example, air bubbles can be detected optically or with ultrasound devices. Air emboli can be detected optically as their absorbance and scattering properties differ from blood in a significant manner. In the infrared spectral region, water has several highly absorbing peaks. Thus, monitoring of wavelengths at these peaks provides good sensitivity to the presence of an air bubble. In the case when a micro-bubble passes, the infrared light is less absorbed and the resulting intensity at the detector increases. Air bubbles can be removed prior to infusion into the patient can be by bubble traps or other filter mechanisms. Alternatively, the bubble can be routed to a waste line to clear it from the infusion circuit. In such a waste line embodiment, the system can continue operation without a requirement of pump stoppage.

Occlusion Detection and Management. The detection of vascular occlusion on either a withdrawal or an infusion can be important for patient safety. Some embodiments of the present invention can determine an occlusion by pressure monitoring or by examination of the sensor response. If fluid flow is unexpectedly stopped or slowed, the sensor response can change for multiple reasons such as heating.

Blood Gas Measurement Capability. Arterial blood gases are important measures of the cardio-pulmonary status of the patient. The measurement of blood gases can be complicated by gas exchanges that result in an inaccurate sample. The transport of a blood sample for the measurement of a blood gas through a stretch of tubing can be problematic due to out gassing or gas exchange with the tubing. Additionally, the surface area associated with the tubing can alter the blood gas as the blood travels though the tube. A blood access system according to the present invention can be more effectively used for blood gas measurement by providing a means for compensation for such effects.

Mechanisms for providing an accurate blood gas measurement can include the use of very short tubing lengths, allowing for equilibration of the blood with the tubing, minimizing the amount of out gassing by the tubing, compensation algorithms to account for changes, or a combination thereof. In the case of a loop system embodiment, the tubing can become equilibrated with the blood. In a second example embodiment, the amount of blood withdrawn can be large enough that the sample measured at the end of the draw has undergone minimal change. Another example embodiment measures the blood gases over the entire sample draw with a projection to an equilibrated point. Different blood draw mechanisms or operating parameters can be used for glucose measurements than are used for blood gas measurements. For example, equilibration concerns can indicate that a larger volume of blood be drawn for blood gas measurements than is required for glucose measurements.

Minimization of Blood-Saline Mixing. Blood-saline mixing can be undesirable in most applications of the present invention. Blood-saline mixing generally means either more saline infused to the patient (if more of the blood is to be returned) or greater blood loss (if less of the saline is to be infused). Generally, blood pushes saline better than saline pushes blood due in part to viscosity differences.

Reduction of shear forces at the wall of the tubing can decrease blood-saline mixing. Tubing with low shear walls can be used. Coating of the tubing with a lubricious substance can significantly reduce the degree of mixing that occurs. Additionally these coatings are often hydrophilic so that shear forces are additionally reduced. Reduction of the distance over which the blood saline junction travels can reduce blood-saline mixing.

Reduction of Circuit Blood Volume. In some applications of the present invention, it can be important to minimize the total amount of blood removed from the body and present in the circuit. For example, the clotting system can become activated when placed in contact with foreign materials. In such applications, a sample can be isolated at a location close to the patient. Any blood beyond that required for the sample can be quickly re-infused to minimize blood residence time. This isolated sample can then be measured without requiring a larger volume of blood to be present in the blood measurement system.

Supplementing Venous Blood Flow. In some applications, the volume of venous blood accessible by the system can be supplemented by use of a standard pressure cuff proximal to the sampling site (e.g., for sampling through access at the lower arm, the cuff might be best positioned at the upper arm). The pressure cuff can be inflated at a preset time period before commencing blood withdrawal, forcing the venous pressure to the cuff pressure, increasing vascular volume, and increasing the available blood flow. As an example, the cuff can be inflated to 40 mmHg or a pressure less than arterial pressure if desired. The cuff can be deflated before commencing infusion, minimizing the back pressure experienced by the system during infusion. A pressure sensor within the circuit can be used as a trigger for the initiating the withdrawal of blood.

As some ICU patients have automatic blood pressure cuffs in place, the system can leverage the increased venous pressure and volume that occurs during the measurement process for the procurement of a blood sample. The operator or the system itself could sense the initiation of an automatic blood pressure measurement by changes in pressure, activation sounds or signals directly from the physiological monitor. For example the GE Dash™ 3000 Patient monitor has an analog blood pressure output that could be utilized for to trigger blood procurement. The blood access system would then utilize the increased venous pressure and associated blood volume due to cuff pressure and procure a blood sample. Such supplementation of the venous blood volume available can help facilitate the procurement of blood samples on a repeatable basis.

Detached Operation. In some applications, it can be desirable to temporarily detach the system from the patient and still use the system for measurement of glucose or other analytes. For example, the system can be prepared for detachment (e.g., by one of the cleaning techniques described herein), and then capped. The patient might then be moved into an operating room, MRI, or CT scanner without the complication of the patient attachment or the need to accommodate the system in the often-crowded room. Blood can be drawn manually in the operating room, carried to the system, and injected into the system for an on-demand glucose reading (e.g., similar to the validation sample method described above). After the reading is taken the blood can be sent to waste. The patient can be reconnected to the system once out of the operating room and sampling resumed.

Sensor Calibration. Almost all types of analyte sensors are subject to drift over time. The ability to periodically calibrate these sensors is often desired and necessary. Within the context of a blood analyte measurement system for use in a setting like an intensive care unit, a simple and easy to use calibration procedure is desired. Such a calibration procedure should minimize nurse intervention and should maintain the overall sterility of the device. The system can provide a calibration point at zero or low analyte concentration as well as a second calibration point at a known analyte concentration or other pre-determined points. Although the example embodiments generally show a two point calibration methodology, the system can be expanded and modified to create more calibration points. The use of multiple calibration points can allow the system to correct for both slope and bias drifts. The system can also be modified to provide one or more validation samples. The calibration process can change over the life of the sensor. Most sensors undergo a “burn in” or conditioning period where more frequent calibration is needed. Over time the sensor typically becomes more stable and the amount and type of calibration may change. For example, a sensor can require a two point calibration initially but after stabilization, only a one point calibration might be needed. The present invention enables a multitude of options in both calibration and validation to ensure effective operation of the system.

A basis for calibration is the use of fluid sources that can be used for calibration. These fluid sources can contain known analyte concentrations and can also contain additional additives that improve the overall performance of the system. Specific additives that can be contained in the fluids include additives that reduce bubble formation, facilitate cleaning of the circuit, reduce protein buildup on the sensing element, reduce cellular aggregation or platelet adhesion to the circuit. As examples, heparin and citrate can be used as additives that reduce the possibility of cellular aggregation. As used in this application; fluid sources, saline fluids, calibration fluids, or maintenance fluids are not intended to be restricted to only normal saline but further include any fluid it that can be administered to patients in environments such as the intensive care unit. Such fluids include but are not limited to normal saline, ½ normal saline, ¼ normal saline, parenteral nutrition, and lactated ringers. Additionally, the fluid source can contain drugs or medications. Specifically, it can be beneficial to include insulin in one of the fluid sources so that the rate of fluid infusion can be used to control the patient's glucose level. In general terms, the saline fluid is the fluid used to maintain the patency of the access site and will typically serve as the low analyte calibration level. The calibration fluid is typically considered as a secondary fluid designed specifically to facilitate calibration or the overall operation of the device. As exposure of the sensor to calibration fluid is critical for operation, one of the fluid sources can include a visible indicator so the operator can see the fluid in contact with the sensor. An example dye is indocyanine green. Indocyanine green is a sterile, water-soluble dye that is used clinically as a dilution indicator for studies involving the heart, liver, lungs, and circulation. These general terms are not intended to be restrictive but to provide a better context for the following descriptions.

Those skilled in the art will realize that the sensor can be attached to a microprocessor system. This system can provide the user with use information including instructions, visual or audible clues associated with the calibration process. Such information can be used to indicate completion of the calibration or measurement, effective cleaning, etc

An important advantage of some embodiments of a blood analyte measurement system according to the present invention is the ability to perform sensor recalibration in a completely sterile manner. Infection risks within intensive care unit patients are extremely high. Some embodiments of the present invention can provide a calibration procedure that does not require “opening” of the system to potential bacteria.

Sensor Sample State. The sensor has the ability to provide feedback on the sample state in the sensor. For example, the sensor can be used to help determine if the sample is undiluted. As the fluid sample moves from saline to an undiluted sample, the sensor output will increase until a stable level. Examination of the output can provide information of the arrival of an undiluted sample. The analyte sensor or other faster response sensors can be used for the determination of an undiluted sample. Potassium is an example sensor that can be used due to a fast response and the fact that potassium is not present in most IV fluids. The cleaning of the system can also be examined by the sensor in a similar manner.

The following figures illustrate a number of example embodiments of the present invention. Each example embodiment generally provides one or more of the desired attributes of the blood analyte measurement system as described above. For purposes of this disclosure, a fluid selection device will encompass any device that allows the user to select a designated fluid source or to stop fluid flow. Such a device can also have the ability to control flow rate from a fluid source. Some fluid selection devices enable selection of a fluid path that enables the removal or addition of fluid, for example by a syringe. A variety of flow selection devices can be used with the preferred embodiments, including but not limited to stop cocks (two way, three way, four way, etc.), pinch valves, butterfly valves, ball valves, rotating pinch valves and linear pinch valves, cams and the like. In some embodiments, a flow selection device selects the fluid source to be used and controls the flow rate from the fluid source.

As used in the disclosure a flow generation device controls the flow of fluids within the system by creating pressure gradients or allowing existing gradients to be transmitted such that fluid flow occurs. In some example embodiments, a flow generation device is configured to regulate the exposure of the sensor to the fluid sources inluding calibration fluids and blood from the host. In some example embodiments, the flow generation device is depicted as a syringe, but can include valves, cams, pumps, and the like. In one example embodiment, the flow generation device is a peristaltic pump. Other suitable pumps include volumetric infusion pumps, peristaltic pumps, and piston pumps. Flow generation devices also include any mechanism that creates a needed pressure gradient for operation. Such a pressure gradient can be generated by varying the pressure at the fluid source by raising/lowering the fluid source. Additionally pressure gradients can be created by placement of pressure cuff around a fluid source (typically an IV bag) or through the use of any mechanism that creates a pressurized bag.

As used in the following embodiments, a fluid source is any source of fluid used in the operation of the blood analyte measurement system. These fluid sources can be used for calibration, cleaning, verification and maintenance of the system. The fluid sources can contain known analyte concentrations and can also contain additional additives that improve the overall performance of the system. Specific additives that can be contained in the maintenance fluid include additives that reduce bubble formation, facilitate cleaning of the circuit, reduce protein buildup on the sensing element, reduce cellular aggregation or platelet adhesion to the circuit. As examples, heparin and citrate are known anticoagulants that reduce cellular aggregation. As used in this description fluid sources can include saline fluids or maintenance fluids can include any fluid it that is commonly administered to patients in environments such as the intensive care unit. Such fluids can include but are not limited to normal saline, ½ normal saline, and lactated ringers. In general terms, the saline fluid is the fluid used to maintain the patency of the access site. The calibration fluid is typically considered as a secondary fluid designed specifically to facilitate calibration or the overall operation of the device. These general terms are not intended to be restrictive but to provide a better context for the following descriptions.

Some of the example embodiments use a reservoir for fluid storage. A reservoir as used in this description includes any device that allows for the storage of a variable volume of fluid. Examples include but are not limited to a bag, a flexible pillow, a syringe, a bellows device, a device that can be expanded through pressure, an expandable fluid column, etc.

As shown in some of the example embodiments the flow generation device and reservoir can be combined into a single system, referred to as the flow generation and reservoir system. An example of such a system is a syringe which has both flow generation and reservoir capabilities. A syringe or syringe pump is defined broadly as a simple piston pump consisting of a plunger that fits tightly in a tube or container. The plunger can be pulled and pushed along inside a cylindrical tube (the barrel) or container, allowing the syringe to take in and expel a liquid. Such syringe systems for procurement of blood are used in clinical practice. Known syringe systems include Deltran Plus Needleless Arterial Blood Sampling System, VAMP Venous Arterial blood Management Protection, Portex Line Draw Plus, Becton Dickinson Safedraw, Smiths Saf-T Closed Blood Collection System, and Hospira SafeSet Closed Blood Sampling system (the foregoing are claimed as trademarks by their respective owners). Another example is a standard peristaltic pump coupled with a reservoir to provide both flow generation and reservoir capabilities.

As shown in some example embodiments, there is a waste channel such as a fluid pathway to a waste bag. During the blood withdrawal process, the fluid volume withdrawn can be transferred into a reservoir, returned to one of the fluid sources, or transferred to waste. For infection control purposes and to minimize contamination, it is typically undesirable to return the fluid volume to any of the fluid sources. Such a process can dilute a calibration at a fixed analyte concentration or add glucose or other analytes to a solution containing no analytes. Additionally, the potential introduction of red blood cells or other cellular matter results in contamination of the fluid source. If no reservoir is used and the fluid is not returned to a fluid source, then the fluid displaced by the withdrawal process can be transferred to a waste channel. One way valves can be used to ensure one way flow into the waste bag and out of the fluid source(s). Such unidirectional flows ensure that contamination does not occur

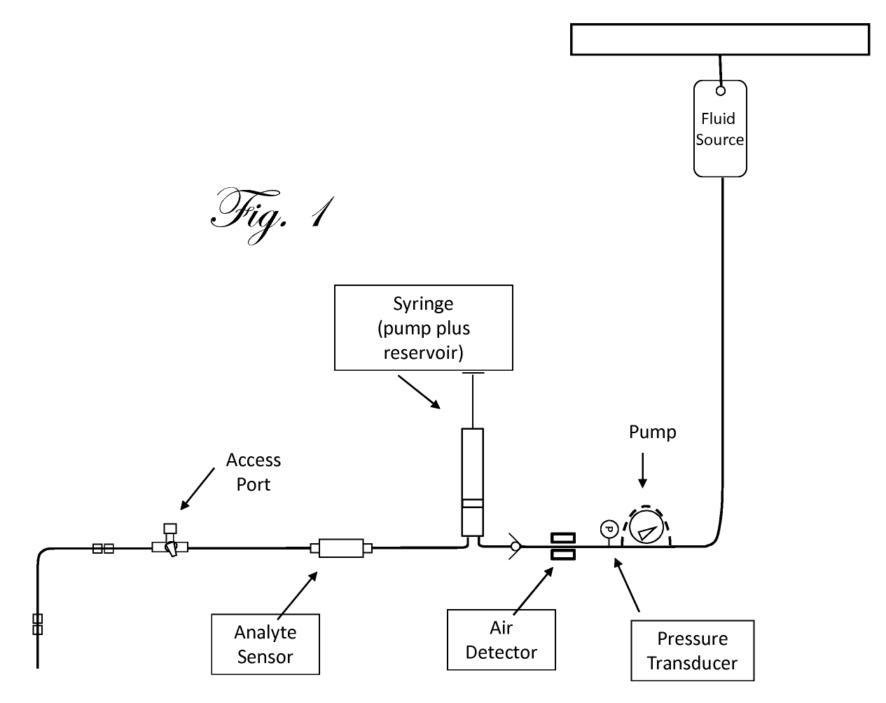

Example Embodiment: Push-pull system using syringe and peristaltic pump. FIG. 1 is a schematic depiction of an example embodiment of the present invention having a syringe push-pull operation. A syringe is used as a flow generation device. The syringe creates a pressure gradient to withdraw blood from the patient to the sensor. Additionally, the syringe serves as a reservoir since the initial blood present will be mixed with saline. Following completion of the measurement, the syringe can be pushed to remove all fluid from the cylinder. Additional washing of the system can be provided by the peristaltic blood pump shown. The example embodiment comprises: a blood access point, a measurement sensor, a needle-less access port, a syringe, a pressure measurement device, a peristaltic pump, and a saline or calibration bag. The operation of the example embodiment is described below.

Blood sample and measurement process:

1. The syringe is used to initiate the draw by moving the plunger away from the home position. The draw continues until an undiluted sample is present at the measurement sensor.
2. The blood interacts with measurement sensor and an analyte measurement is made.
3. Following completion of the measurement, the syringe is pushed towards the home position so that the blood is returned to the patient.
4. Following the return of the syringe to the home position, the pump is activated so as to move saline or calibration fluid through the system to the patient. This process helps clean the circuit and remove any remaining blood in the circuit.
5. Following cleaning of the circuit, the blood pump may remain active to maintain a “keep vein open” fluid infusion towards the patient.
6. The measurement results and any historical information are communicated to a user, e.g., shown on a display (not shown).

The example embodiment of FIG. 1 can provide several important characteristics:

1. Analyte measurements can be made on a very frequent basis.
2. The system operates with no blood loss.
3. The system operates with very little saline infusion and only during cleaning.
4. The system can work on multiple access locations, including arterial.
5. The system contains a pressure monitor that can provide arterial, central venous, or pulmonary artery catheter pressure measurements after compensation for the pull and push of the blood access system.
6. The system can compensate for different size catheters through the volume pulled via the syringe.
7. The system provides for a one point calibration via the saline or calibration bag.
8. The system provides for access to the blood sample via a port in the circuit.

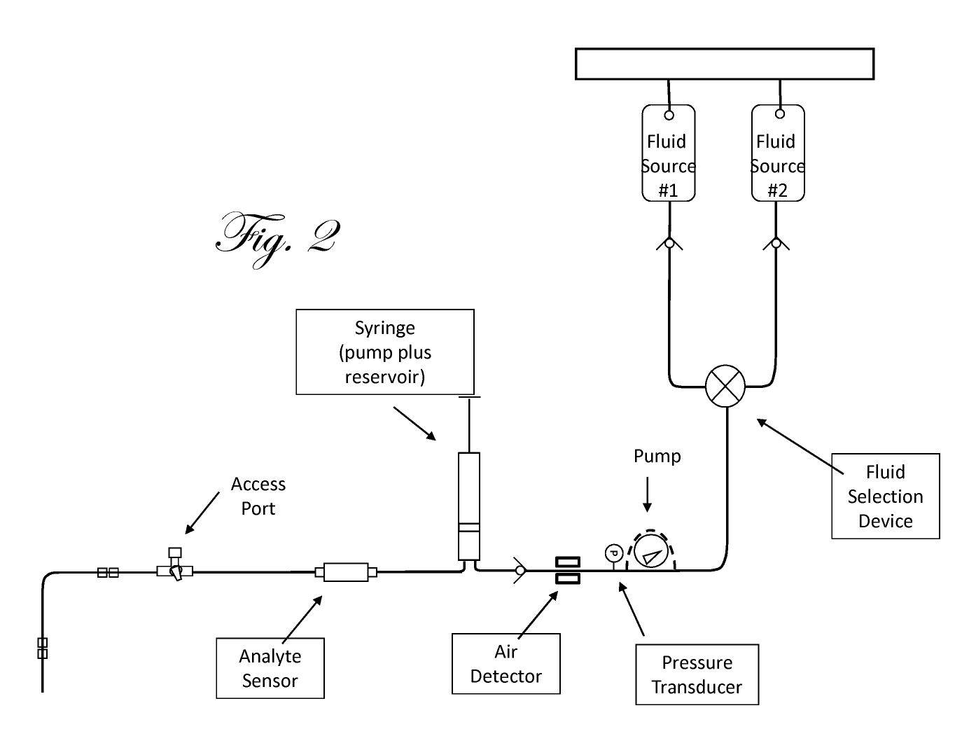

Example Embodiment: Push Pull System Based upon Syringe and Peristaltic Pump with Two Point Calibration. FIG. 2 is a schematic depiction of an example embodiment of the present invention having a syringe push-pull operation. In the example embodiment, the flow generation device shown is a syringe. The syringe creates a pressure gradient to withdraw blood from the patient to the sensor. Additionally, the syringe serves as a reservoir since the initial blood present will be mixed with saline. Following completion of the measurement, the syringe is pushed to remove all fluid from the cylinder. The system has the ability to perform a two point calibration via selection of the fluid source by the flow selection device. Additional washing of the system is provided by the peristaltic blood pump shown. The system comprises: a patient interface device such as catheter or other blood access point to the patient, a measurement sensor in fluid communication with the patient interface device, a needle-less access port in fluid communication with the sensor, a syringe in fluid communication with the needle-less access port, a pressure measurement device in fluid communication with the syringe, a peristaltic pump in fluid communication with the syringe, a fluid selection valve in fluid communication with the peristaltic pump and, through individual one-way valves, with two fluid bags that can contain two separate calibration fluids. The operation of the example embodiment is described below.

Blood sample and measurement process:

1. The syringe initiates the draw by moving the plunger away from the home position. The draw continues until an undiluted sample is present at the measurement sensor.
2. The blood interacts with measurement sensor and an analyte measurement is made.
3. Following completion of the measurement, the syringe is pushed towards the home position so that the blood is returned to the patient.
4. Following the return of the syringe to the home position, the pump is activated so as to move saline or calibration fluid through the system to the patient. This process helps clean the circuit and removed any remaining blood in the circuit.
5. Following cleaning of the circuit, blood pump may remain active to maintain a “keep vein open” fluid infusion towards the patient.
6. The measurement results and any historical information are communicated to a user, e.g., shown on a display (not shown).

Calibration process. The system has two fluid sources that can be used to facilitate calibration of the sensor. The fluid sources have different glucose levels. The fluid selection device can be used to select the fluid of choice. The peristaltic pump can then move the fluid so that the sensor is exposed to the designated calibration fluid. The pump may remain active during this period and flow calibration fluid over the sensor pump may stop and allow the calibration fluid to simply remain in contact with the sensor.

The example embodiment of FIG. 2 can provide several important characteristics:

1. The system can provide a two point calibration of sensor.
2. Analyte measurements can be made on a very frequent basis.
3. The system operates with no blood loss.
4. The system requires very little saline infusion and only during cleaning.
5. The system can work on multiple access locations including but not limited to arterial.
6. The system contains a pressure monitor that can provide arterial, central venous, or pulmonary artery catheter pressure measurements after compensation for the pull and push of the blood access system.
7. The system can compensate for different size catheters through the volume pulled via the syringe.
8. The system provides for a one point calibration via the saline or calibration bag.
9. The system provides for access to the blood sample via a port in the circuit.

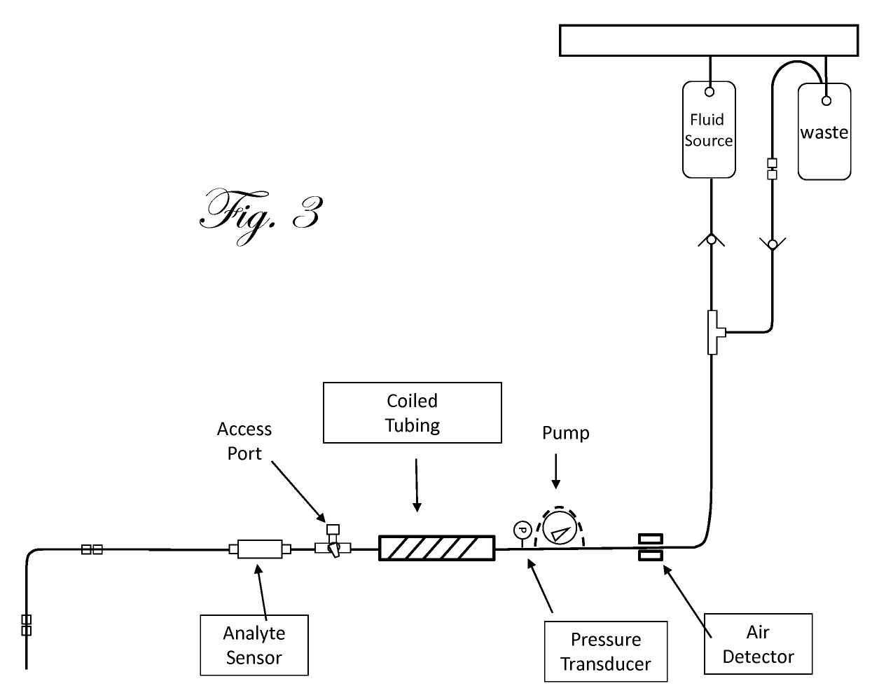

Example embodiment: Push-Pull System Based upon Tubing Reservoir and Peristaltic Pump. FIG. 3 is a schematic depiction of an example embodiment of the present invention having a push-pull operation with a fluid pathway to divert fluid to waste. The system prevents possible red blood cell lysis by ensuring that no blood enters the peristaltic pump. The system provides for storage of the blood-saline junction in a tubing coil. The system prevents any contamination of the saline bag by diverting the withdrawal fluid into a waste bag. The system has appropriate occlusion detection via pressure monitoring, blood access via an access port, provides flow control during the measurement process, and the use of the peristaltic pump permits pulsed or variable wash sequences. The system comprises: a blood access point to the patient, a measurement sensor, a needle-less access port, tubing coil, a pressure measurement device, a peristaltic pump, a t-junction, a fluid bag for calibration with a one-way valve allowing fluid flow from the fluid bag to the t-junction, and a waste bag with a one-way valve allowing fluid flow from the t-junction to the waste bag. As one of skill on the art would appreciate, a second calibration fluid or multiple calibration fluids can be added in a manner similar to that described in FIG. 2. The operation of the example embodiment is described below.

Blood sample and measurement process:

1. Peristaltic pump initiates the draw by moving blood toward the sensor. The draw continues until an undiluted sample is present at the measurement sensor.
2. The blood interacts with measurement sensor and an analyte measurement is made.
3. Following completion of the measurement, the peristaltic pump infused the blood back into the patient.
4. Following the return of the blood to the patient, the pump is activated so as to move saline or calibration fluid through the system to the patient for additional cleaning. This process helps clean the circuit and removed any remaining blood in the circuit.
5. Following cleaning of the circuit, blood pump may remain active to maintain a “keep vein open” fluid infusion towards the patient.
6. The measurement results and any historical information are communicated to a user, e.g., shown on a display (not shown).

The example embodiment of FIG. 3 can provide several important characteristics:

1. The system is fully automatic system and does not require nurse intervention.
2. Analyte measurements can be made on a very frequent basis.
3. The system operates with no blood loss.
4. The system requires very little saline infusion and only during cleaning.
5. The system can work on multiple access locations including arterial.
6. The system contains a pressure monitor that can provide arterial, central venous, or pulmonary artery catheter pressure measurements after compensation for the pull and push of the blood access system.
7. The system can compensate for different size catheters through the volume pulled via the syringe.
8. The system provides for a one point calibration via the saline or calibration bag.
9. The system provides for access to the blood sample via a port in the circuit.

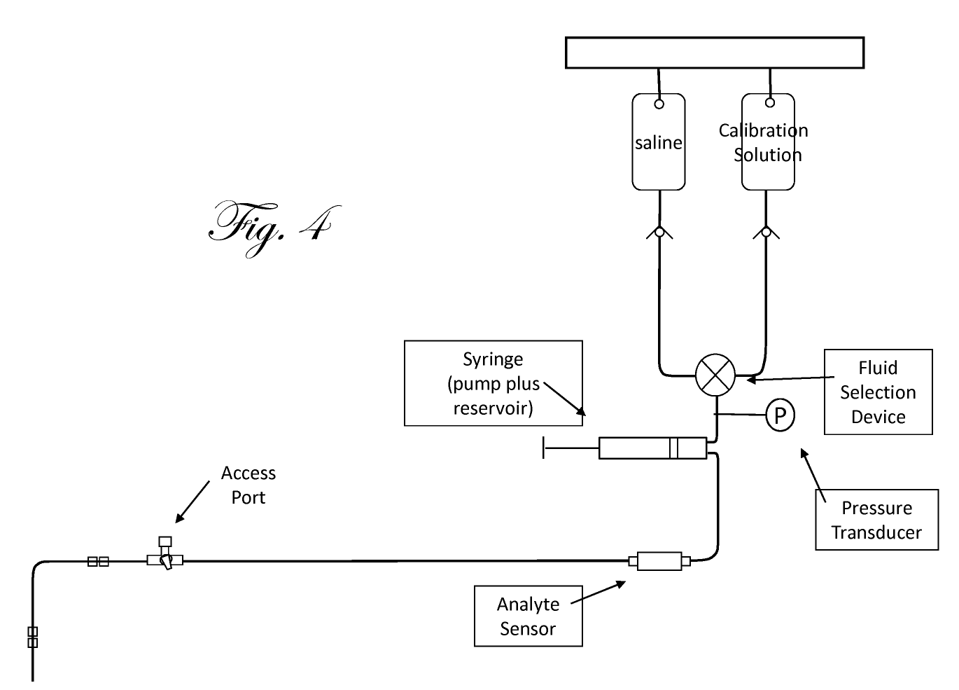

Example embodiment: Push Pull System Based upon Syringe. FIG. 4 is a schematic depiction of an example embodiment of the present invention with a sensor close to a reservoir. The example embodiment can be described as a push pull system where the flow generation device is a syringe. The syringe creates a pressure gradient to withdraw blood from the patient to the sensor. The system as shown is manually operated. The syringe serves as a reservoir as the initial blood present will be mixed with saline. The use of a reservoir as shown eliminates the need for a separate waste bag. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, the syringe is pushed so as to remove all fluid from the cylinder. Additional washing of the system is provided by allowing flow from the fluid sources towards the patient. In the case of venous attachment, this flow can be by gravity. The system comprises: a catheter providing access patient, a stopcock or other access port, a measurement sensor, a syringe, a pressure measurement device, a fluid selection device allowing selection of the fluid sources and fluid sources for maintenance and calibration of the system. One-way valves can be mounted with the bags to allow fluid flow from the bags to the fluid selection device. The operation of the example embodiment is described below.

Blood sample and measurement process:

1. The system is calibrated as described below. Following calibration the operator initiates a blood draw by moving the syringe plunger away from the home position. The draw continues until an undiluted sample is present at the measurement sensor. The determination of an undiluted sample can be by volume drawn, visual inspection or the sensor sample state methods described above.
2. The blood interacts with measurement sensor and an analyte measurement is made. The blood can be flowing or not flowing across the sensor during the measurement.
3. Following completion of the measurement, the syringe is pushed towards the home position so that the blood is returned to the patient. At this juncture the majority of all blood has been returned to the patient.
4. If additional cleaning of the circuit is desired, fluid from either fluid source can be used to clean the circuit further. The fluid can simply be flowed through the system or drawn into the syringe. If drawn into the syringe, the operator can use a push-stop-push flow pattern to facilitate cleaning. The cleaning process helps to maintain the circuit for future use and prevent clotting of the circuit.
5. Following cleaning of the circuit, fluid for may continue to flow toward the patient to create a “keep vein open” fluid infusion towards the patient.
6. The measurement results and any historical information are communicated to a user, e.g., shown on a display (not shown).

Calibration process. The system has two fluid sources that can be used to facilitate calibration of the sensor. The fluid sources have different analyte levels. The fluid selection device can be used to select one of the two fluids. Gravity feed or pressure moves the fluid so that the sensor is exposed to the designated calibration fluid. During the calibration process, calibration fluid can be flowed over the sensor or fluid may simply remain in contact with the sensor. As described elsewhere in this specification it can be advantageous to maintain the sensor in a low analyte containing solution prior to measurement.

The example embodiment of FIG. 4 can provide several important characteristics:

1. Analyte measurements can be made on a very frequent basis.
2. The system operates with no blood loss.
3. The system can work on multiple access locations including arterial.
4. The system contains a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port.
9. The system provides completely sterile operation.

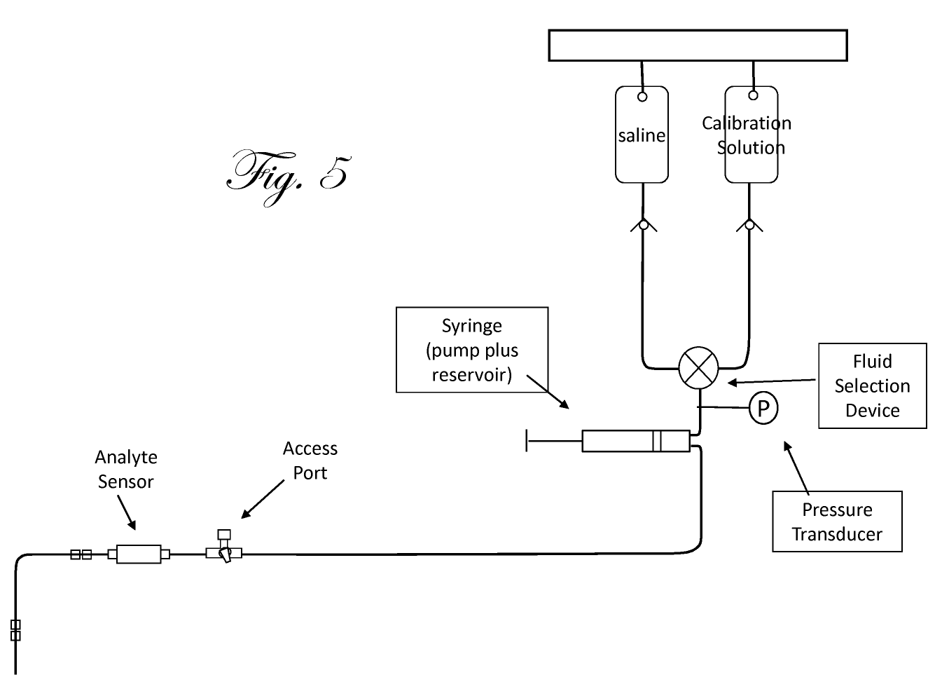

Example embodiment: Push Pull system based upon Syringe with Sensor Near Patient. FIG. 5 is a schematic depiction of a push pull system based upon a syringe and is very similar to FIG. 4. A difference between the two example embodiments is the location of the sensor. In FIG. 5 the sensor is located very close to the patient. The location of the sensor close to the patient reduces the blood draw volume needed to get an undiluted sample to the sensor. The syringe creates a pressure gradient to withdraw blood from the patient to the sensor. The operational characteristics of the example embodiment of FIG. 5 are very similar to FIG. 4.

FIG. 5 is a push pull system using a syringe as a flow generation device. Prior to initiation of a measurement, the system allows for maintenance of the sensor in a low glucose concentration fluid. To initiate a measurement, the syringe creates a pressure gradient to withdraw blood from the patient to the sensor. The system as shown is manually operated. The syringe serves as a reservoir since the initial blood present will be mixed with saline. The use of a reservoir as shown eliminates the need for a separate waste bag. The system has the capability of doing a two point calibration as described below. The access port shown allows for procurement of a blood sample or the introduction of additional calibration, validation or check samples. The pressure measurement device allows for pressure monitoring. If attached to an arterial line the fluid bags can be pressurized to create a pressure gradient to create positive flow to the patient. The system operates in an entirely sterile manner. Following completion of the measurement, the syringe can be pushed to remove all fluid from the syringe cylinder. Additional washing of the system can be provided by allowing flow from the fluid sources towards the patient. In the case of venous attachment, this flow can be by gravity.

For calibration, the system can use two fluid sources with different glucose concentrations. The fluid selection device can be used to select the fluid of choice, or a controlled combination of fluids. Gravity feed or pressure moves the fluid so that the sensor is exposed to the designated calibration fluid. During the calibration process, calibration fluid can be flowed over the sensor or calibration fluid can simply remain in contact with the sensor. Following calibration the sensor can be exposed to a low glucose containing solution prior to measurement.

FIG. 5 is a schematic illustration of a blood access system using a single access line. The system comprises: a catheter providing access patient, a stopcock or other access port, a measurement sensor, a syringe, a pressure measurement device, a fluid selection device allowing selection of the fluid sources for maintenance and calibration of the system.

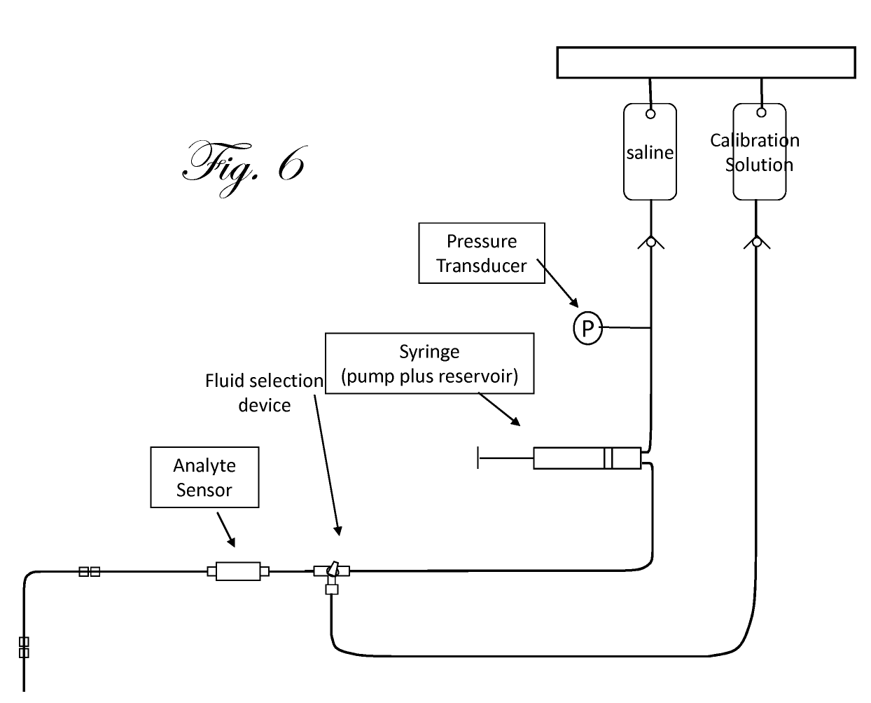

Example embodiment: Push Pull system based upon Syringe with Calibration Fluid Pathway FIG. 6 is a schematic illustration of an example embodiment comprising a push pull system based upon a syringe. The syringe creates a pressure gradient to withdraw blood from the patient to the sensor. The system as shown is manually operated. The syringe serves as a reservoir as the initial blood present will be mixed with saline. The use of a reservoir as shown eliminates the need for a separate waste bag. The system has the capability of doing a two point calibration. The system contains a separate fluid pathway with a connection near the sensor. This separate fluid path helps to minimize the amount of calibration solution that is infused into the patient. To effectively expose the sensor to a calibration fluid, the stopcock needs to be opened the sensor exposed to the calibration fluid. The short length of tubing reduces mixing and the total volume of fluid needed. An additional port on the existing stopcock or an additional stopcock or port (not shown) allows for procurement of a blood sample or the introduction of additional calibration, validation or check samples. The pressure measurement device allows for pressure monitoring. The pressure measurement system can be attached to the either fluid pathway and in operation must be exposed to the pressure changes of the patient for effective pressure measurement. If attached to an arterial line the fluid bags would be pressurized to create a pressure gradient to create positive flow to the patient. The system is closed to the environment and operates in an entirely sterile manner. Following completion of the measurement, the syringe is pushed so as to remove all fluid from the cylinder. Additional washing of the system is provided by allowing flow from the fluid sources towards the patient. In the case of venous attachment, this flow is by gravity. The system comprises: a catheter providing access patient, a stopcock or other access port, a measurement sensor, a fluid connection to the calibration fluid, a syringe, a pressure measurement device, a stopcock allowing selection of the fluid sources and fluid sources for maintenance and calibration of the system. One-way valves can be mounted with the system to allow fluid flow from the bags to the system. The operation of the example embodiment is described below.

Blood sample and measurement process.

1. The system is calibrated as described below. Following calibration the operator initiates a blood draw by moving the syringe plunger away from the home position. The draw continues until an undiluted sample is present at the measurement sensor. The determination of an undiluted sample can be by volume drawn, visual inspection or the sensor sample state methods described above.
2. The blood interacts with measurement sensor and an analyte measurement is made. The blood may be flowing or not flowing across the sensor during the measurement.
3. Following completion of the measurement, the syringe is pushed towards the home position so that the blood is returned to the patient. At this juncture the majority of all blood has been returned to the patient.
4. If additional cleaning of the circuit is desired, fluid from either fluid source can be used to clean the circuit further. The fluid can simple by flowed through the system or drawn into the syringe. If drawn into the syringe, the operator can use a push-stop-push flow pattern to facilitate cleaning. The cleaning process helps to maintain the circuit for future use and prevent clotting of the circuit.
5. Following cleaning of the circuit, fluid can continue to flow toward the patient to create a “keep vein open” fluid infusion towards the patient.
6. The measurement results and any historical information are communicated to a user, e.g., shown on a display (not shown).

Calibration Process. The system has two fluid sources that can be used to facilitate calibration of the sensor. The fluid sources have different analyte levels. The fluid selection device can be used to select the fluid of choice. Several different methods can be used to move the fluid over the sensor. As an example, gravity feed can move the fluid so that the sensor is exposed to the designated calibration fluid. As another example, the fluid sources can be pressurized to move the fluid. As another example, additional flow generation devices can be added to create flow. As shown in FIG. 6, the syringe in combination with the flow selection device can be used to pull fluid from the fluid sources with subsequent flow occurring over the sensor. The calibration solution is delivered via the bypass circuit to the sensor. During the calibration process calibration fluid can be flowed over the sensor or fluid can simply remain in contact with the sensor. Following calibration of the sensor with the calibration fluid, the fluid selection device is configured to select the saline fluid. As described elsewhere in this specification it can be advantageous to maintain the sensor in a low analyte containing solution prior to measurement. Based upon these advantages and the general desire not to infuse the patient with high analyte concentration fluid, the higher analyte containing solution would be the calibration solution. The saline solution can be simply saline, other IV fluids, an IV fluid with anticoagulant, or a calibration solution with a lower analyte value.

The example embodiment of FIG. 6 can provide several important characteristics:

1. Analyte measurements can be made on a very frequent basis.
2. The system operates with no blood loss.
3. The system can work on multiple access locations including arterial.
4. The system can contain a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port (not shown).
9. The system provides completely sterile operation.
10. The use of the calibration bypass circuit helps to limit the amount of calibration solution infused into the patient.

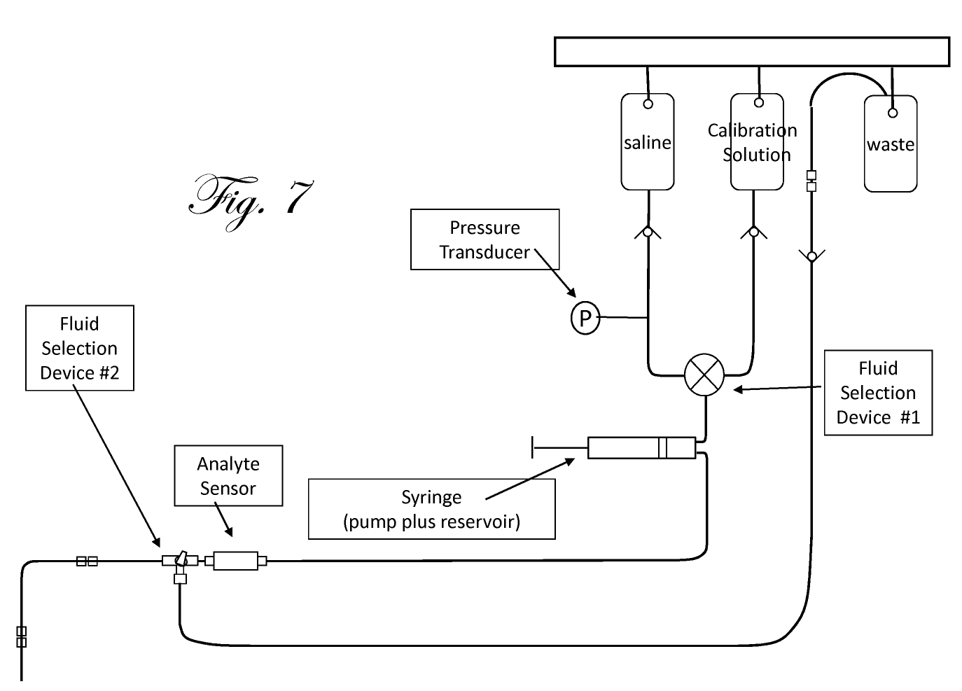

Example embodiment: Push Pull system based upon Syringe with Waste Fluid Pathway. FIG. 7 is a schematic illustration of an example embodiment comprising a push pull system based upon a syringe. The syringe creates the pressure gradient needed to withdraw blood from the patient to the sensor. The system is shown is manually operated. The syringe serves as a reservoir as the initial blood present will be mixed with saline. The use of a reservoir as shown eliminates the need for a separate waste bag. The system has the capability of doing a two point calibration. The system contains a separate fluid pathway to the waste bag. This separate fluid path helps to minimize the amount of solution that is infused into the patient. For example, all fluid used for calibration and or cleaning can be directed to waste bag. Fluid selection device number one is used to define the fluid flowing to the sensor. If the operator desires to have the fluid directed to waste, fluid selection device number #2 can position such that fluid flow is to waste bag. The use of fluid selection device #2 coupled with the waste bypass pathway provides the operator with the opportunity of moving all calibrate and/or waste fluids to the waste bag. An additional port on the existing stopcock or an additional stopcock or port (not shown) allows for procurement of a blood sample or the introduction of additional calibration, validation or check samples. The pressure measurement device allows for pressure monitoring. The pressure monitoring system can be attached to any of the fluid pathways shown provided that in operation the pressure measurement system has appropriate exposure to the pressure variations from the patient. If attached to an arterial line the fluid bags can be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. This additional washing fluid can be infused into the patient or directed to the waste bag. In the case of venous attachment, this flow can be by gravity. The system comprises: a catheter providing access patient, a stopcock or other access port, a measurement sensor, a fluid connection to the waste bag, a syringe, a pressure measurement device, a stopcock allowing selection of the fluid sources and fluid sources for maintenance and calibration of the system. One-way valves can be mounted with the system to allow fluid flow from the fluid bags to the system, and to allow fluid to flow from the system to the waste bag. The operation of the example embodiment is described below.

Blood sample and measurement process.

1. The system is calibrated as described below. Following calibration the operator initiates a blood draw by moving the syringe plunger away from the home position. The draw continues until an undiluted sample is present at the measurement sensor. The determination of an undiluted sample can be by volume drawn, visual inspection or the sensor sample state methods described above.
2. The blood interacts with measurement sensor and an analyte measurement is made. The blood may be stagnant during the measurement process or flowing across the sensor.
3. Following completion of the measurement, the syringe is pushed towards the home position so that the blood is returned to the patient At this juncture the majority of all blood has been returned to the patient. At any point during the infusion process, the operator may elect to direct the fluid to waste.
4. If additional cleaning of the circuit is desired, fluid from either fluid source can be used to clean the circuit further. The fluid used for cleaning can be directed to waste by fluid selection device #2. The fluid can flow through the system or be drawn into the syringe. If drawn into the syringe, the operator can use a push-stop-push flow pattern to facilitate cleaning. The cleaning process helps to maintain the circuit for future use and prevent clotting of the circuit.
5. Following cleaning of the circuit, fluid can continue to flow toward the patient to create a “keep vein open” fluid infusion towards the patient.
6. The measurement results and any historical information are communicated to a user, e.g., shown on a display (not shown).

Calibration Process. The system has two fluid sources that can be used to facilitate calibration of the sensor. The fluid sources have different glucose levels. The fluid selection device can be used to select the fluid of choice. Gravity feed moves the fluid so that the sensor is exposed to the designated calibration fluid or alternatively, the fluid sources can be pressurized to move the fluid. The calibration solution is delivered to the sensor and either he infused into the patient or directed to the waste bag. During the calibration process, calibration fluid may be flowed over the sensor or fluid may simply remain in contact with the sensor. Following calibration of the sensor with the calibration fluid, the fluid selection device #1 is configured to select the saline fluid. As described elsewhere in this specification it can be advantageous to maintain the sensor in a low analyte containing solution prior to measurement. Based upon these advantages and the general desire not to infuse the patient with high analyte concentration fluid, the higher analyte containing solution would be the calibration solution. The saline solution can be simply saline, other IV fluids, an IV fluid with anticoagulant, or a calibration solution with a lower analyte value.

The example embodiment of FIG. 7 can provide several important characteristics:

1. Analyte measurements can be made on a very frequent basis.
2. The system operates with no blood loss.
3. The system can work on multiple access locations including arterial.
4. The system can contain a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port (not shown).
9. The system provides completely sterile operation.
10. The use of the waste bypass pathway helps to limit the amount of solution infused into the patient.

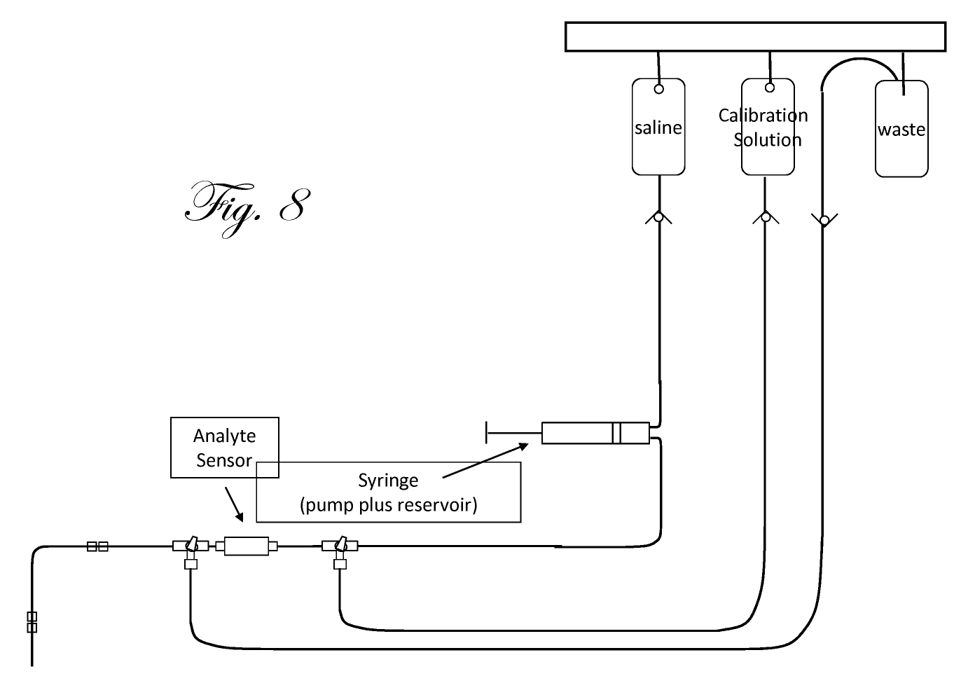

Example embodiment: Push Pull system based upon Syringe with Calibration and Waste Fluid Bypass Circuits. FIG. 8 is a schematic illustration of an example embodiment that combines characteristics of the example embodiments illustrated in FIGS. 6 and 7. The system is push pull based via the use of a syringe. The syringe creates the pressure gradient needed to withdraw blood from the patient to the sensor. The system is shown is manually operated. The syringe serves as a reservoir as the initial blood present will be mixed with saline. The use of a reservoir as shown eliminates the need for a separate waste bag. The system has the capability of doing a two point calibration. The system contains two separate fluid pathways. The first is between the calibration solution and a fluid selection device in fluid connectivity with the sensor. The second pathway is between the waste bag and a second fluid selection device in fluid connectivity with a sensor. These separate fluid paths can be used to minimize the amount of solution that is infused into the patient. An additional port on the existing stopcock or an additional stopcock or port (not shown) allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. This additional washing fluid can be infused into the patient or directed to the waste bag. In the case of venous attachment, this flow is by gravity. The system comprises: a catheter providing access patient, a stopcock or other access port, a measurement sensor, a fluid connection to the calibration bag, a fluid connection to the waste bag, a syringe, a pressure measurement device, a stopcock allowing selection of the fluid sources and fluid sources for maintenance and calibration of the system. One-way valves can be mounted with the system to allow fluid flow from the fluid bags to the system, and from the system to the waste bag.

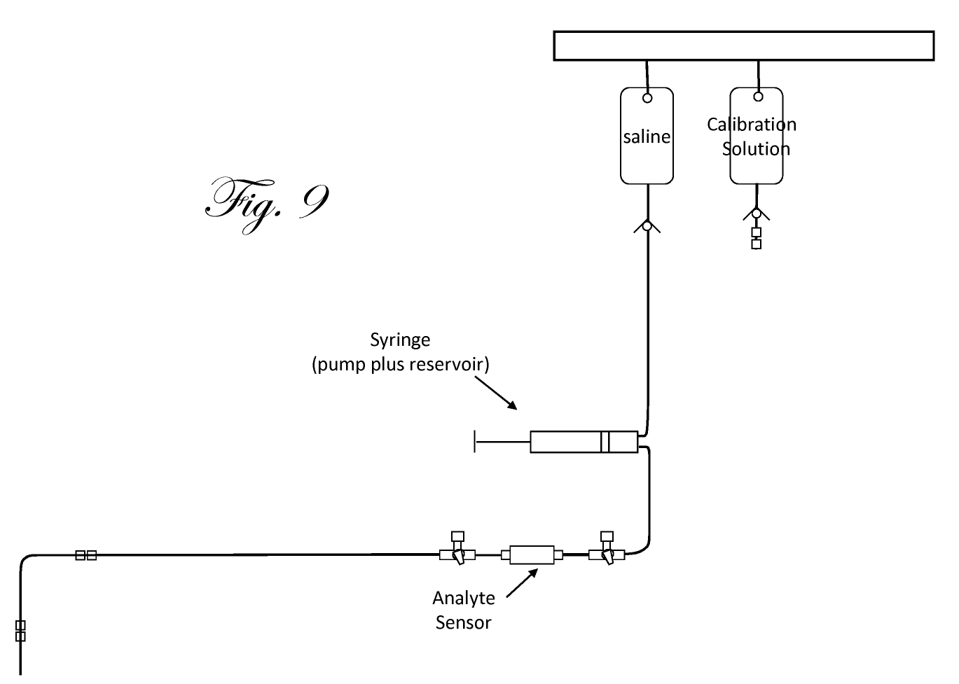

Example embodiment: Push Pull System Based upon Syringe with Sensor Access. FIG. 9 is a schematic illustration of an example embodiment comprising a push pull system based upon a syringe. The syringe creates the pressure gradient needed to withdraw blood from the patient to the sensor. The system as shown is manually operated. The syringe serves as a reservoir as the initial blood present will be mixed with saline. The use of a reservoir as shown eliminates the need for a separate waste bag. The system has the capability of doing a one, two or multi-point calibration. The system contains two fluid selection devices located on either side of the sensor. These fluid selection devices provide fluid access sites that can be used to calibrate the sensor, procure blood samples, and run additional validation samples separate. As an example, two syringes can be attached to the two fluid selection devices shown. Fluid can be transferred from one syringe to the other such that flow occurs over the sensor. Such a manual process can have advantages in quality control and the amount of fluid infused into the patient. The existing ports or an additional stopcock or port (not shown) allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. This additional washing fluid can be infused into the patient or directed to the waste bag. In the case of venous attachment, this flow is by gravity. The system comprises: a catheter providing access patient, two fluid selection devices, a measurement sensor, a syringe, a pressure measurement device, and fluid sources for maintenance and calibration of the system. One-way valves can be mounted with the system to allow fluid flow from the fluid bags to the system.

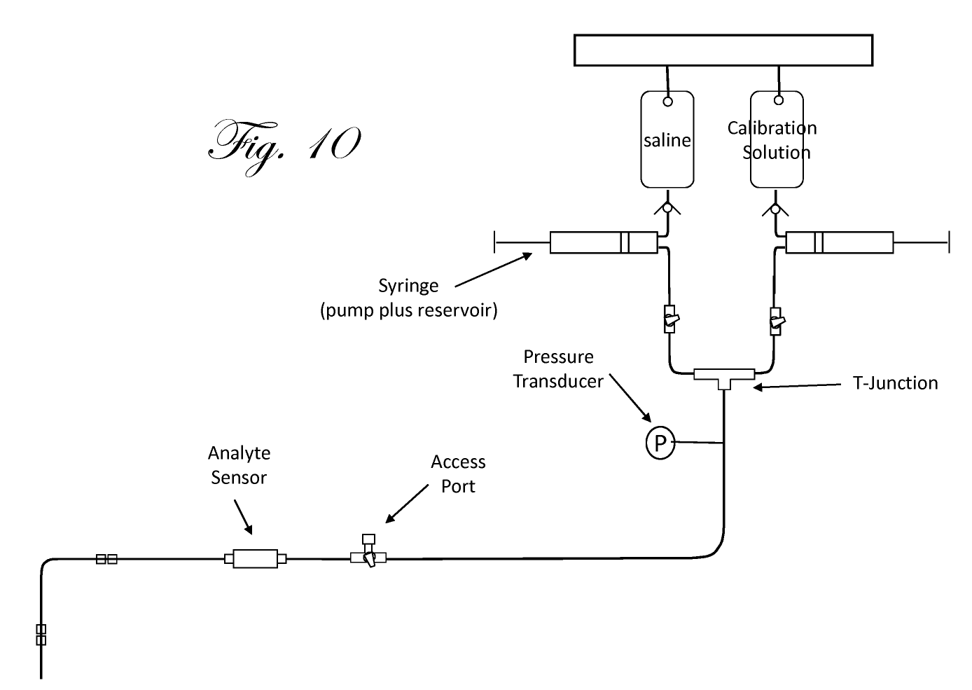

Example embodiment: Two Syringe Push Pull System. FIG. 10 is a push pull system based upon two syringes. The syringes create the pressure gradient needed to withdraw saline or blood away from the patient to the sensor. The system is shown is manually operated. The syringe serves as a reservoir as the initial blood present will be mixed with saline. The use of a reservoir as shown eliminates the need for a separate waste bag. The two syringes provide flexibility in operation. For example, only saline could be pulled into a first syringe while mostly blood is pulled into a second syringe. Such a division of blood and saline might limit the amount of anticoagulant needed to prevent clotting. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, the syringe is pushed so as to remove all fluid from the cylinder. Additional washing of the system is provided by allowing flow from the fluid sources towards the patient. In the case of venous attachment, this flow is by gravity. The two syringes can be used individually or in combination to facilitate cleaning of the system. The system comprises: a catheter providing access patient, a stopcock or other access port, a measurement sensor, a T-junction, a pressure measurement device, two syringes, and appropriate check and fluid sources for maintenance and calibration of the system. One-way valves can be mounted with the system to allow fluid flow from the fluid bags to the system.

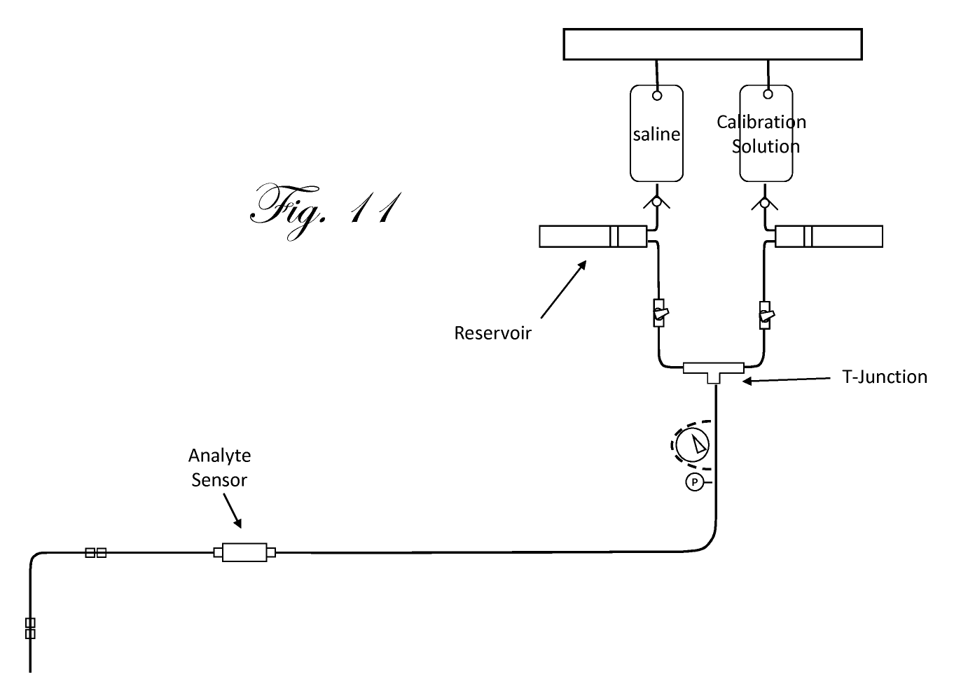

Example embodiment: Two Reservoir Push Pull System with Peristaltic Pump. FIG. 11 is a schematic illustration of an example embodiment comprising an automated system using two reservoirs and a pumping mechanism. The pump creates the pressure gradient needed to withdraw saline or blood away from the patient to the sensor. The fluid withdrawn can be directed into one or two available reservoirs. The use of a reservoir(s) as shown eliminates the need for a separate waste bag. If two reservoirs are utilized, they provide flexibility in operation. For example, only saline could be pulled into one reservoir while mostly blood is pulled into the other reservoir. Such configuration might limit the amount of anticoagulant needed to prevent clotting. The system has the capability of doing a two point calibration. The valves shown allow the operator to select the associated fluid pathway. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. In the case of venous attachment, this flow is by gravity. The pump can be operated to facilitate cleaning of the system. The system comprises: a catheter providing access to a patient, a stopcock or other access port, a measurement sensor, a pump, a pressure measurement device, a T-junction, two reservoirs, two valves, appropriate check (one-way) valves and fluid sources for maintenance and calibration of the system.

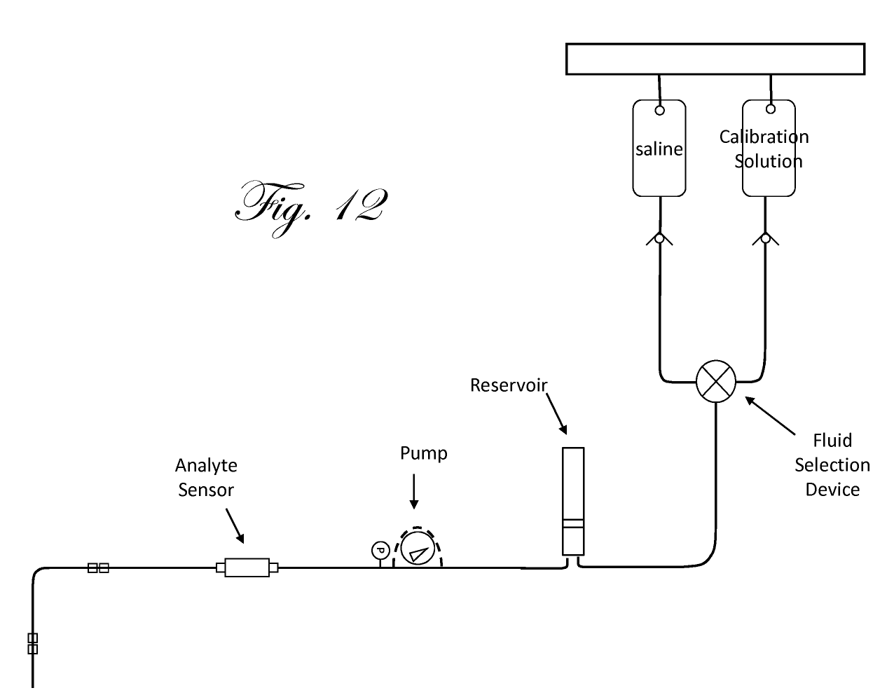

Example embodiment: Push Pull System based upon Peristaltic Pump. FIG. 12 shows a push pull system based upon a peristaltic pump. The system configuration is similar to FIG. 4 except that the pressure gradient for flow is provided by a pump. The pump creates a pressure gradient to withdraw blood from the patient to the sensor. The blood reservoir serves as a reservoir as the initial blood present will be mixed with saline. The use of a reservoir as shown eliminates the need for a separate waste bag. The system has the capability of doing a two point calibration. The pressure measurement device allows for pressure monitoring. If attached to an arterial line the pump can create the appropriate pressure gradient needed to enable fluid infusion. The system operates in an entirely sterile manner. Following completion of the measurement, the pump is activated to push the blood towards the patient. Additional washing of the system can be provided by the pump, specifically the pump can provide a stop-push or back and forth cleaning action.

FIG. 12 is a schematic illustration of a blood access system using a single access line. The system comprises: a catheter providing access patient, a pump, a measurement sensor, a reservoir, a pressure measurement device, a fluid selection device allowing selection of the fluid sources and fluid sources for maintenance and calibration of the system. One-way valves can be mounted with the system to allow fluid flow from the fluid bags to the system.

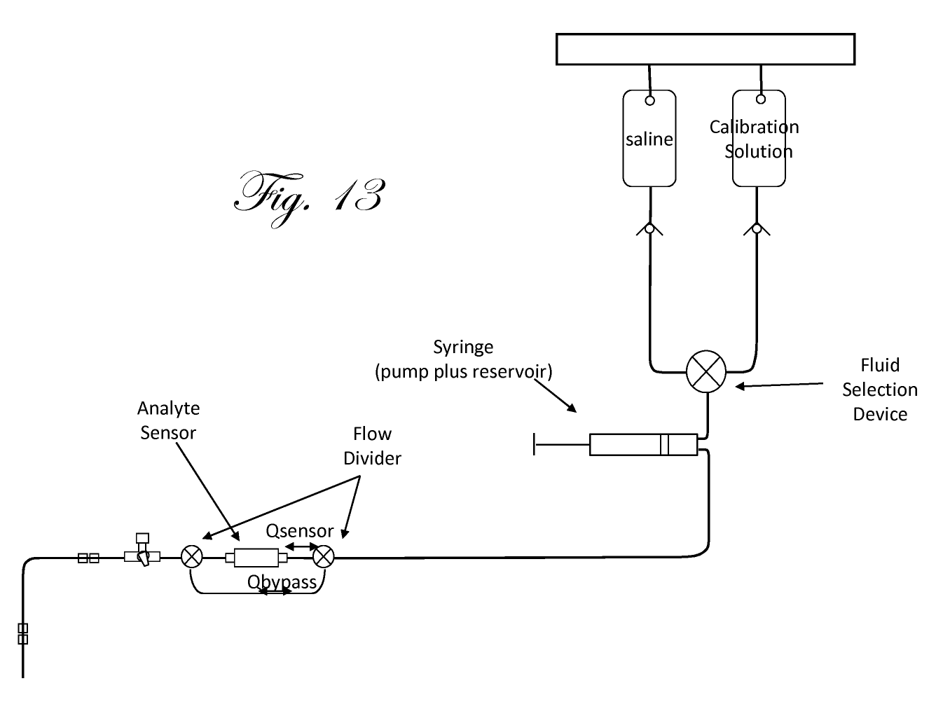

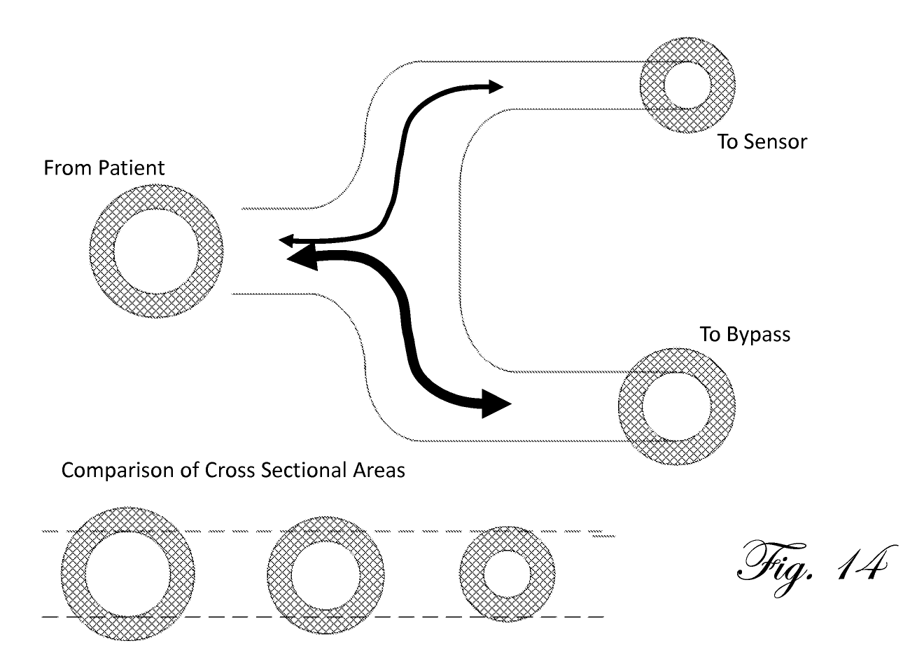

Example embodiment: Push Pull System Based upon Syringe with Flow Divider Bypass. FIG. 13 is a schematic illustration of an example embodiment comprising a push pull system where the flow generation device is a syringe. The syringe creates the pressure gradient needed to withdraw blood from the patient to the sensor. The system is shown is manually operated. The syringe serves as a reservoir as the initial blood present will be mixed with saline. The system also contains a bypass configuration intended to limit the flow rate through sensor during the filling and reinfusion phases. The slower flows through the sensor limit the shear caused by flow through the small diameter of the sensor. The flow divider is designed to divide the flow between the two channels in a manner that allows for a good measurement and cleaning of the sensor while concurrently limiting the shear stress on the blood and sensor. One possible embodiment uses different cross sectional areas to provide the appropriate flow resistance to achieve the above goals. See FIG. 14 for an example flow divider. The use of a reservoir as shown eliminates the need for a separate waste bag. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. In the case of venous attachment, this flow is by gravity. The system comprises: a catheter providing access patient, a stopcock or other access port, a flow divider, a measurement sensor, a syringe, a pressure measurement device, a fluid selection device allowing selection of the fluid sources and fluid sources for maintenance and calibration of the system. One-way valves can be mounted with the system to allow fluid flow from the fluid bags to the system.

FIG. 14 is a schematic illustration of a flow divider. The cross section areas of the three tubes are sized so that appropriate flow and associated sheer is achieved through the sensor. The lower part of FIG. 14 shows the different cross sectional areas.

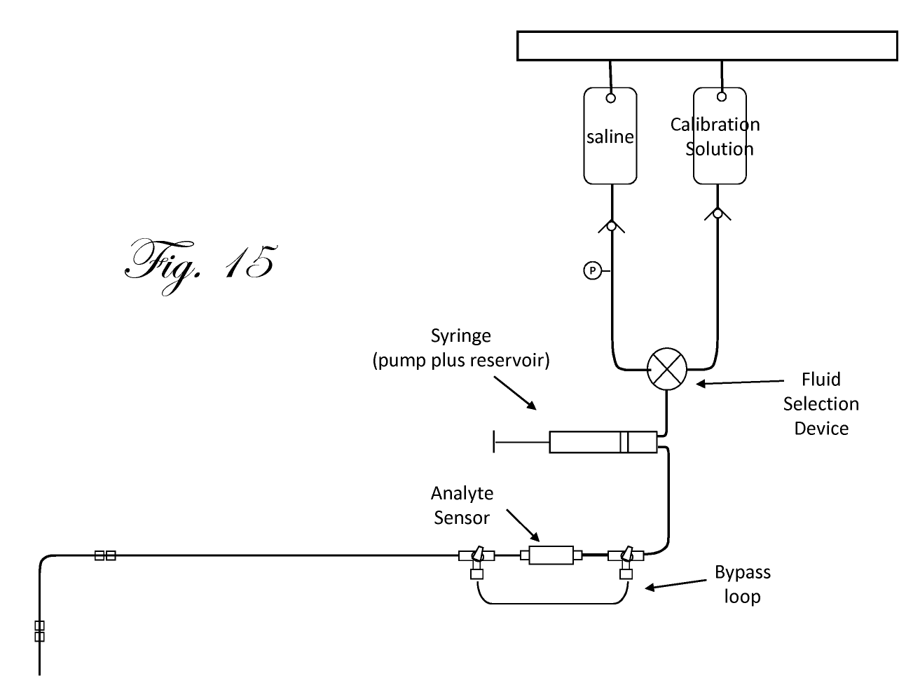

Example embodiment: Push Pull System Based upon Syringe with Flow Divider Bypass. FIG. 15 is a schematic illustration of an example embodiment comprising a push pull system where the flow generation device is a syringe. The syringe creates the pressure gradient needed to withdraw blood from the patient to the sensor. The system is shown is manually operated. The syringe serves as a reservoir as the initial blood present will be mixed with saline. The system also contains a bypass configuration which allows flow to be diverted around the sensor. For the reduction of shear within the sensor, it may be desirable to bypass during periods of maximum flow periods. Additionally, the bypass is configured with stopcocks on either side of the sensor to allow user to put the sensor in-line for measurement phase, then isolate the sensor from the circuit to prevent sensor-related disruption of the blood pressure signal. The use of a reservoir as shown eliminates the need for a separate waste bag. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. In the case of venous attachment, this flow is by gravity. The system comprises: a catheter providing access patient, a stopcock or other access port, a flow divider, a measurement sensor, a syringe, a pressure measurement device, a fluid selection device allowing selection of the fluid sources and fluid sources for maintenance and calibration of the system. One-way valves can be mounted with the system to allow fluid flow from the fluid bags to the system.

The example embodiment of FIG. 15 can provide several important characteristics:

1. Analyte measurements can be made on a very frequent basis.
2. The system operates with no blood loss.
3. The system can work on multiple access locations including arterial.
4. The system contains a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port (not shown).
9. The system provides completely sterile operation.
10. If the sensor has a small cross sectional area or significant compliance, then the bypass circuit enables pressure monitoring without corruption of the signal during non-measurement periods.
11. If the sensor has a small cross sectional area or can be damaged by flow, then the bypass circuit can be used. In practice, an undiluted sample could be drawn to the sensor location via the bypass loop. At this point in the measurement cycle, the fluid selection devices changes to flow through the sensor occurs. The additional blood needed to fill the sensor is small in comparison the amount needed to get an undiluted sample to the sensor.

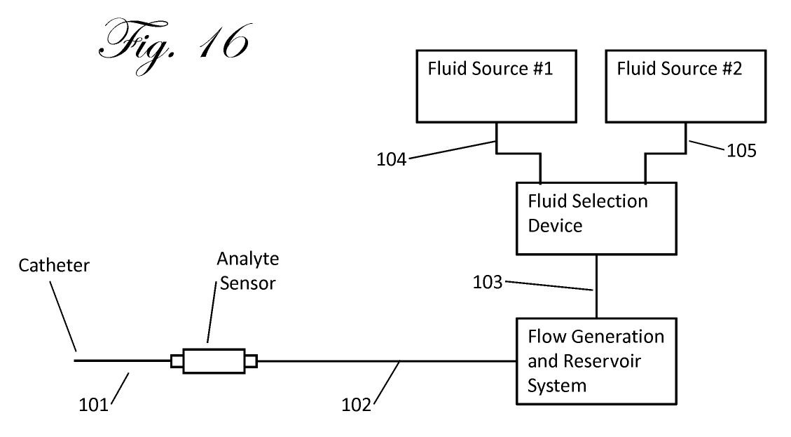

Example embodiment: system configuration. FIG. 16 is a block diagram of an example embodiment. The system comprises a catheter (or similar blood access device) suitable to be placed in fluid communication with the vascular system of a patient, and in fluid communication with an analyte sensor via a first fluid transport apparatus 101. A second fluid transport apparatus 102 connects the analyte sensor with the flow generation and reservoir system. A third fluid transport apparatus 103 connects the flow generation device with a fluid selection device. The fluid selection device is connected to one of more fluid sources via fourth 104 and fifth 105 fluid transport apparatuses. The flow generation and reservoir system can be a single device such as a syringe or can include separate devices such as a pump and bag. In operation, the flow generation device uses the first fluid transport apparatus to draw blood from the patient to the analyte sensor. Fluid exits the sensor into the second fluid transport apparatus. The fluid is moved by the flow generation device and stored in the fluid reservoir. The operator can use the flow generation device to flow blood over the sensor during the measurement, or measurements can be made with the fluid in a stagnant state. Following completion of the measurement the flow generation device infuses the withdrawn fluid into the patient. Additional cleaning can be conducted as needed. The example embodiment has the ability to conduct a two point calibration by using the fluid selection device. The fluid selection device can be used to select the desired fluid source to enable calibration of the sensor. Multiple methods and fluid sequences can be used for calibration within the context of the example embodiment. As examples of such calibration, see U.S. patent application Ser. No. 12/576,303 “Method for Using Multiple Calibration Solutions with an Analyte Sensor with Use in an Automated Blood Access System” filed Oct. 9, 2009, incorporated herein by reference. When the system is not making a measurement or being calibrated, the flow generation device in combination with the flow selection device can be used to flow a fluid source through first and second fluid transport apparatuses toward the patient to maintain open access to the circulatory system of the patient.

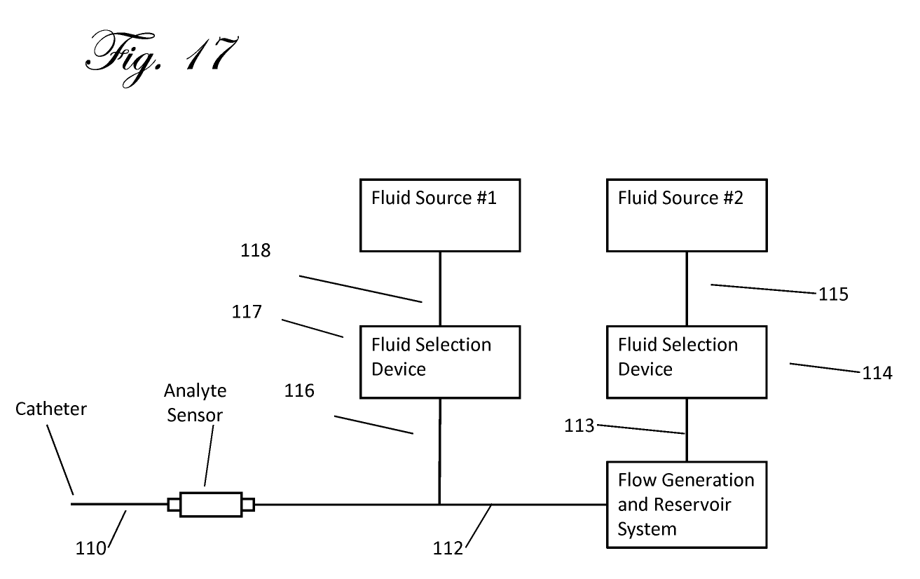

Example embodiment: system configuration. FIG. 17 is a block diagram of an example embodiment. The system comprises a catheter (or similar blood access device) suitable to be placed in fluid communication with the vascular system of a patient, and in fluid communication with an analyte sensor via a first fluid transport apparatus 110. A second fluid transport apparatus 112 connects the analyte sensor with the flow generation and reservoir system. A third fluid transport apparatus 113 connects the flow generation and reservoir system with a fluid selection device 114. The fluid selection device is connected to a fluid source #2 via a fourth fluid transport apparatus 115. A fifth fluid transport apparatus 116 connects fluid selection device 117 to fluid transport apparatus 112. A sixth fluid transport apparatus 118 connects the fluid selection device 117 to a fluid source #1. The flow generation and reservoir system can be a single system such as a syringe or can include separate devices such as a pump and a bag. In operation, the flow generation device uses the first fluid transport apparatus to draw blood from the patient to the analyte sensor. Fluid exits the sensor into the second fluid transport apparatus. The fluid is moved by the flow generation device and stored in the fluid reservoir. The operator can use the flow generation device to flow blood over the sensor during the measurement, or measurements can be made with the fluid not flowing. Following completion of the measurement the flow generation device infuses the withdrawn fluid into the patient. Additional cleaning can be conducted as needed. The example embodiment has the ability to conduct a two point calibration by using the fluid selection devices 117 and 114. Fluid selection device 117 can be configured (e.g., opened to fluid flow) so the analyte sensor is exposed to fluid source #1. Fluid selection device 114 can be configured (e.g., opened to fluid flow) to provide the sensor access to fluid source #2. The fluid selection devices can be used to select the desired fluid source to enable calibration of the sensor. Multiple methods and fluid sequences can be used for calibration within the context of the example embodiment. As examples of such calibration, see U.S. patent application Ser. No. 12/576,303 “Method for Using Multiple Calibration Solutions with an Analyte Sensor with Use in an Automated Blood Access System” filed Oct. 9, 2009, incorporated herein by reference. When the system is not making a measurement or being calibrated, the flow generation device in combination with the flow selection device can be used to flow a fluid source through first and second fluid transport apparatuses toward the patient to maintain open access to the circulatory system of the patient.

Calibration and Maintenance. The present invention can also provide improved methods for maintaining and calibrating an analyte sensor such as a glucose sensor for improved performance and safety. Via recognition of enzyme kinetics, the improved methods facilitate a faster measurement response which limits the potential for blood coagulation. The improved methods also reduce enzyme suppression which can lead to inaccurate results. The improved methods, via the use of a low glucose concentration maintenance fluid, create a safer system by limiting the potential for erroneously high readings. The risk of infection associated with an automated glucose system can be decreased through the use of a low glucose concentration maintenance glucose solution.

In the case of automated blood measurements, it is desirable to minimize the amount of time needed for generation of an accurate measurement result. The potential for blood coagulation increases with the amount of time the sample is removed for the patient's vascular system. Coagulation is a complex process by which blood forms clots for the maintenance of hemostasis (the cessation of blood loss from a damaged vessel). Coagulation begins almost instantly after exposure of the blood to a non-endothelium (lining of the vessel) surface. Coagulation is a multi-step process involving the release phospholipid components called tissue factor and fibrinogen. These initiate a chain reaction resulting eventually in a platelet plug or clot. The process is time dependent, thus the longer the exposure the greater the likelihood of clotting.

The coagulation process can be minimized or stopped by the addition of external agents, for example heparin. The ability to simultaneously anti-coagulate blood and make accurate measurements creates a measurement accuracy dilemma. In general terms, the measurement process should be made on an undiluted sample or a sample with a very accurately defined amount of dilution. A defined amount of dilution creates additional complexity to the system. Therefore, a common practice is to use a heparinized solution. The solution helps to prevent clotting after the measurement is completed and by coating the tubing of the system. The actual sample being measured has no or almost no dilution by heparin. Therefore, heparin helps to prevent coagulation but coagulation can occur if the sample is removed from the vascular system for a period of time as the actual sample sees little or no heparin.

From the perspective of an automated blood glucose monitoring system, coagulation is undesirable. Coagulation can lead to clotting/occlusion of the access site. The creation of a complete or partial restriction can prevent blood flow while a partial occlusion can result in increased draw pressures and longer blood transport times. Coagulation can lead to the infusion of clots and subsequent embolic damage. As noted above, coagulation is a multi-step process which is generally triggered by a member of the clotting cascade. A potential risk is that the clotting cascade becomes activated due to the exposure to a non-endothelium surface and embolic events occur in the patient. The issue of coagulation is especially important when thinking about one intended use population: intensive care patients. These patients often exhibit hyper-coagulation states. Therefore, any automated system should seek to minimize measurement time, the time the sensor is in contact with blood, for the minimization of coagulation and the inherent risks associated with coagulation.

One approach to minimizing measurement time is to ensure that the sensor is maintained in a solution that does not limit reaction time or lead to the general suppression of sensor response. The maintenance solution is the solution in contact with the sensor prior to a measurement. The maintenance solution can be, but is not required to be, part of the calibration process. The use of maintenance solutions that contain glucose concentrations higher than that of the blood sample to be measured can result in longer measurement times and the general suppression of the sensor.

The use of a high glucose concentration in the maintenance solution, as is required with one point calibration systems, leads to a slower sensor response following exposure of the sensor to a different glucose concentration. In practice the glucose sensor is constructed with a diffusion control membrane that covers the enzyme, typically glucose oxidase. The sensor will respond with different reaction characteristics due to the relationship between the glucose concentration in the maintenance fluid and glucose concentration in the fluid to be measured, the patient's blood. Maintaining the sensor at an elevated glucose concentration will result in the enzyme reaction kinetics approaching a zero order phase. In this zero order phase, the enzyme is saturated and operating at a maximum rate which is generally described as Vmax in the Michaelis-Menton equation. In this condition a change in the glucose concentration has a delayed response due to small changes in the reaction rate of the enzyme.

If the concentration of glucose in the maintenance solution is reduced to zero or a low value in the solution for a period of time, the diffusion of glucose and reaction products will occur through the membrane from the area around the enzyme. The enzyme under these conditions will return to a first order reaction kinetics phase where the rate of reaction is proportional to the concentration of the substrate, glucose. The introduction of a sample with increased glucose concentration will result in an increased reaction rate for the enzyme until the saturation concentration of glucose is attained. At this point the enzyme has again returned to zero order phase reaction kinetics, Vmax. The rate of response of the sensor is directly related to the diffusion of glucose across the membrane covering the enzyme and the concentration of enzyme in the sensor that can react with the glucose. The rate of diffusion of reaction end products across the membrane can differ considerably from the rate of glucose diffusion. Due to the reaction kinetics and the diffusion rates, the sensor response is not symmetric. The response time of the sensor from a low to high glucose concentration is faster than the response time associated with going from a high concentration to a low concentration. Thus, the reaction kinetics dictate that faster response times are possible by using a maintenance solution whose glucose concentration is below that in the blood samples to be measured.

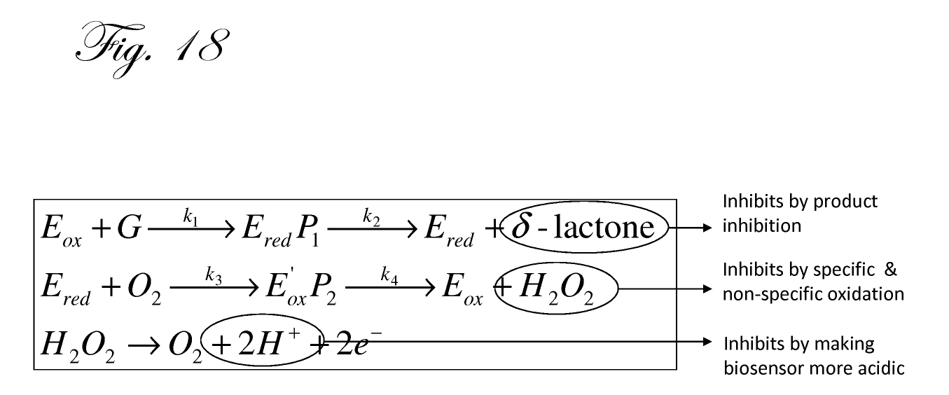

In addition to the reaction kinetics issues described above, the exposure of glucose biosensors using glucose oxidase enzyme to high glucose concentration leads to depressed sensor activity. At least three different, but related, products of the enzymatic cycle are responsible for this reduced activity: glucono-d-lactone, H₂O₂, and acid. FIG. 18 shows the specific reactions. “Kinetics and Mechanism of Action of Glucose Oxidase” by Gibson et al. (1964), The Journal of Biological Chemistry 239, 11, 3927 describes a series of kinetic experiments and kinetic coefficient calculations associated with the enzyme suppression. Subsequent work by Bao et al, “Competitive inhibition by hydrogen peroxide produced in glucose oxidation catalyzed by glucose oxidase”, Biochemical Engineering Journal, (2003), journal 13, 69-72. A review of the available literature defines a variety of mechanism for enzyme activity suppression. Glucono-d-lactone inhibits the reaction by product inhibition. Hydrogen peroxide (H₂O₂) inhibits via competitive inhibition. Acid can cause overall sensor suppression though the creation of an acidic environment as well as influencing the diffusion characteristics of the membrane covering the enzyme. When subjecting the sensor to a lower glucose concentration, a portion of these sensor inhibiting end products must exit across the membrane. Due to variations in size, charge and polarity, these end products can exit at slower rates and thus can present diffusion problems associated with glucose equilibrium. As the enzyme suppressing compounds described above are present in relation to the glucose concentration present in the maintenance solution, low glucose concentration maintenance fluids can be advantageous for the multiple reasons listed above.

For the purpose of an automated blood glucose monitoring system, the presence of enzyme suppressing compounds leads to increased measurement times and the potential for coagulation as well as inaccurate measurements. Issues of accuracy can occur due to delayed reaction times. A delay in reaction completion can result in the sensor drift during the measurement period resulting in an inaccurate measurement. Some measurement systems use the rate of change of sensor output to calculate concentration. If the effective kinetic response of the sensor is variable, then the use of such rate of change measurements will be inaccurate.

In addition to sensor drift problems, a variable measurement time due to sensor activity suppression causes other problems. For the avoidance of coagulation, it is desirable to maintain movement of the fluid during the measurement process. Thus, a prolonged measurement time results in an inaccurate measurement if the measurement time is fixed or requires a prolonged draw. The inaccurate measurement is problematic by itself. The longer draw time creates also problems with the disposition of the drawn blood, infusion of the sample and cleaning of the system. The system is typically designed for a given draw volume and subsequent cleaning related to this volume. Thus, a larger draw will necessitate more cleaning. Such cleaning activities typically lead to more fluid infusion into the patient which is undesirable. Thus, variable draw volumes due to variable reaction kinetics creates a problem for a cleaning perspective.

The improved methods enable the use of low concentration maintenance fluids while concurrently enabling effective calibration with a second solution of higher glucose concentration. The improved methods enable the effective calibration of the system based upon the ability to expose the sensor to two different glucose concentrations while maintaining the sensor in a condition for a fast and accurate response.

The basis for calibration with the improved methods is the use of one or more fluids that can be used for calibration. These calibration fluids can contain known glucose concentrations and can also contain additional additives that improve the overall performance of the system. Specific additives that can be contained in the calibration fluids include additives that reduce bubble formation, facilitate cleaning of the circuit, reduce protein buildup on the sensing element, reduce cellular aggregation or platelet adhesion to the circuit. As examples, heparin and citrate can be used as additives that reduce the possibility of cellular aggregation. In the case of heparin, it can be added to either a saline bag or a calibration fluid bag. Due to the calcium binding effects of citrate, citrate can be added to either the saline or calibration bag while calcium is added to the other bag. Such a methodology can provide for anticoagulation of the blood while also providing a means for replacing any bound calcium by the direct infusion of calcium during the administration of calibration fluid to keep the access site open. One of ordinary skill in the art will recognize that a number of additional additives can be placed in the calibration fluid for the overall improvement or control of system operation.

As used in this description, saline fluids or calibration fluids are not intended to be restricted to only normal saline but to also include any fluid it that is commonly administered to patients in environments such as the intensive care unit. Such fluids include but are not limited to normal saline, ½ normal saline, and lactated ringers. A maintenance fluid is a fluid that present at the sensor prior to a measurement. The maintenance fluid in some cases can be part of the fluids used for calibration. Typically, the maintenance fluid will be a fluid used to maintain the patency of the access site. Typically, access sites are infused in a “keep vein open” or KVO manner at about 3 to 5 ml/hour. In general terms, the calibration fluid is a secondary fluid designed specifically to facilitate calibration or the overall operation of the device. These general terms are not intended to be restrictive but to provide a better context for the following descriptions.

Clinical accuracy needs often dictate higher levels of performance at low glucose levels, often referred to as hypoglycemia, but linearity of response to high glucose levels is also desired. End-users will expect very accurate measurements at hypoglycemic levels and will also expect good linearity over the range of 50 mg/dl to 500 mg/dl. The ability to tailor the calibration procedure based upon the functional sensitivity of the measurement system is a desired aspect of any calibration system. In practice this may require multiple calibration samples at the low glucose levels and concurrently having calibration samples with high glucose concentrations. The present invention can address these calibration needs by providing the opportunity to use multiple saline-based glucose concentration samples as well as providing the opportunity to create a variety of relative glucose changes in the blood sample. One or more of these calibration solutions can be used for the maintenance fluid. Preferably, the maintenance fluid should have a glucose concentration below the level of the samples to be measured. This relationship facilitates a fast and accurate response of the system.

The improved methods are described herein in the context of example blood access and measurement systems, for convenience of description. The improved methods can also be used in combination with other blood access systems, such as those described in the following applications, each of which is incorporated by reference: U.S. provisional 60/791,719, filed Apr. 12, 2006; U.S. provisional 60/913,582, filed Apr. 24, 2007; PCT application PCT/US06/60850, filed Nov. 13, 2006; U.S. application Ser. No. 11/679,826, filed Feb. 27, 2007; U.S. application Ser. No. 11/679,837, filed Feb. 28, 2007; U.S. application Ser. No. 11/679,839, filed Feb. 28, 2007; U.S. application Ser. No. 11/679,835, filed Feb. 27, 2007; U.S. application Ser. No. 10/850,646, filed May 21, 2004; U.S. application Ser. No. 11/842,624, filed Aug. 21, 2007; U.S. application Ser. No. 12/188,205, filed Aug. 8, 2008; U.S. provisional 60/991,373, filed Nov. 30, 2007; U.S. provisional 61/044,004, filed Apr. 10, 2008; U.S. application Ser. No. 12/108,250, filed Apr. 23, 2008; US provisional 61104252, filed Oct. 9, 2008.

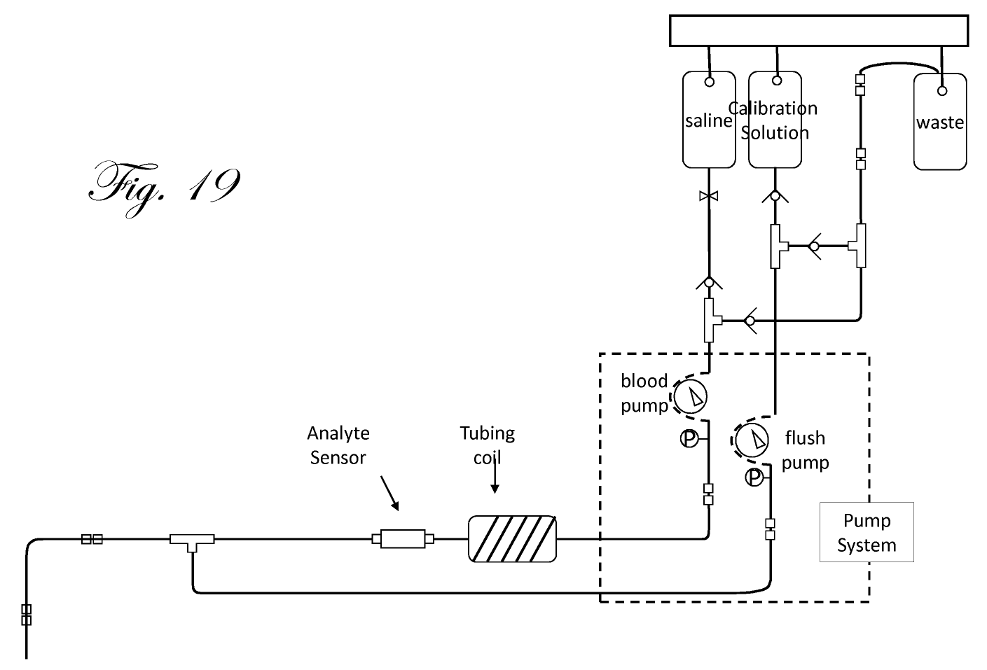

FIG. 19 is an illustration of an example embodiment of a blood access and measurement system suitable for use with the present invention. The example automated blood analyte measurement system contains two fluid bags providing for at least two different calibration points. In use, the analyte sensor can be exposed to a zero or predetermined low glucose concentration via fluid from the saline bag. A second glucose concentration can be provided via fluid from the calibration solution bag. The example system in FIG. 19 provides the opportunity for calibration of the device with a known calibration fluid while concurrently minimizing the infusion of the calibration fluid into the patient. In the example system, the calibration fluid solution can be pumped through the circuit and directly to waste without infusion into the patient. For example, the flush pump can be operated in a manner towards the patient and the blood pump can operate at a similar rate away from the patient. In this manner the analyte sensor is exposed to the calibration fluid solution but no fluid is infused into the patient. Following sensor calibration, fluid from the other fluid bag can be used to wash the circuit in a similar manner. Such a process can enable the effective calibration of the glucose sensor at a second glucose concentration. The system also enables the sensor to be maintained in a solution with low glucose concentration. The system then enables the effective calibration of the system as well as the maintenance of the sensor in a solution that facilitates rapid and accurate results.

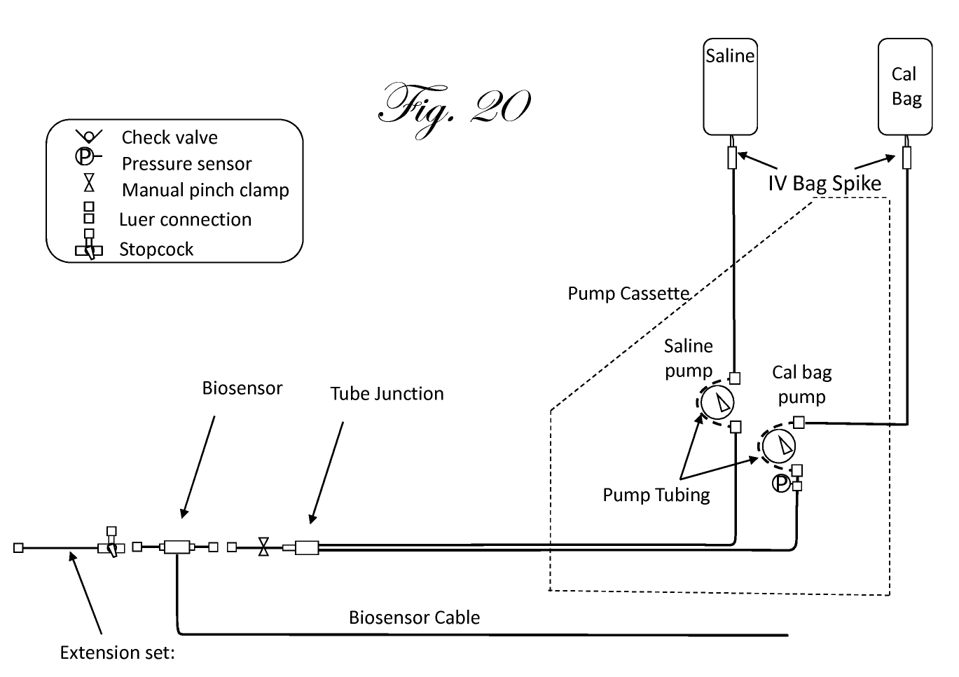

FIG. 20 is an illustration of an example embodiment where the sensor is located near the patient. The sensor can be located in the IV catheter, immediately adjacent to the catheter or generally near the patient. The example automated blood analyte measurement system contains two fluid bags providing for at least two different calibration points, labeled in the figure as saline and cal bag. In use, the analyte sensor can be exposed to a zero or predetermined glucose concentration via fluid from the saline bag. A second glucose concentration can be provided via fluid from the calibration solution bag. The example system in FIG. 20 provides the opportunity for calibration of the device with a known calibration fluid while concurrently minimizing the infusion of the calibration fluid into the patient. In the example system, the calibration solution can be pumped through the circuit so that both tubes going to the sensor are filled with undiluted calibration solution. For example, the cal pump can be operated in a manner towards the patient and the saline pump can operate at a similar rate away from the patient. The fluid would go to waste as needed, (not shown). When the tube junction contains an appropriate calibration solution, the pumps can be activated so as to push the calibration solution to the sensor. The sensor can then be calibrated. To re-fill the circuit with a second calibration solution or a saline without glucose the saline pump can be operated in a manner towards the patient and the cal pump can operate at a similar rate away from the patient. This would result in a second solution near the tube junction. Again the solution can be moved to the sensor by operating both pumps toward the sensor or patient. The total amount of saline infused into the subject is very small when using this “loop” circuit. Such a process enables the effective calibration of the glucose sensor and enables the sensor to be maintained in a low glucose concentration prior to measurement. The location of the sensor near the patient, combined with a method to facilitate fast response from the enzyme sensor, creates a circuit design that can limit the amount of time the blood needs to be out of the body.

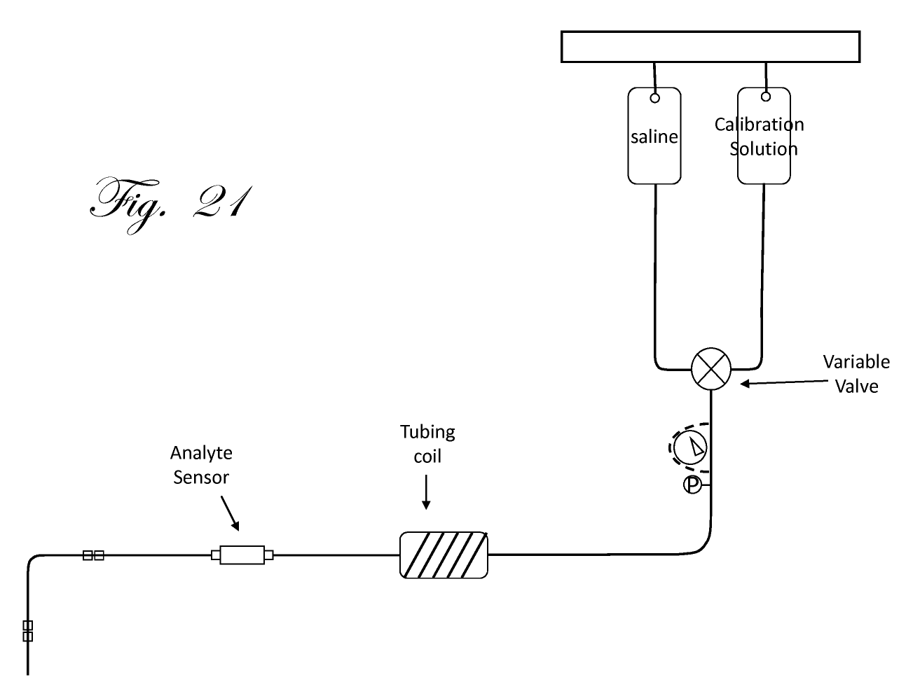

FIG. 21 shows an example implementation of a two level sensor calibration system. The example system in FIG. 21 enables the analyte sensor to be exposed to at least two known glucose concentrations. The variable valve can be a simple stopcock where the solution provided to the analyte sensor is either 100% calibration solution or 100% saline solution. In other embodiments a variable valve can provide for controlled mixing of these two fluid solutions to create multiple glucose concentrations. In any of the envisioned configurations, the system allows for calibration of the sensor and maintenance of the sensor in a low glucose concentration.

The present invention has been described as set forth herein. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1. An apparatus for measuring an analyte in blood taken from a patient, comprising:
a. A patient interface device, capable of interfacing with the circulatory system of a patient;
b. An analyte sensor having first and second ports, with the first port in fluid communication with the patient interface device;
c. A flow generation and reservoir system having first and second ports, with the first port in fluid communication with second port of the analyte sensor; and
d. A first fluid source, mounted such that it can be placed in fluid communication with the second port of the flow generation and storage system, wherein the first fluid source provides a first fluid having a first predetermined analyte concentration.

2. An apparatus as in claim 1, further comprising a second fluid source mounted such that it can be placed in fluid communication with the second port of the flow generation and reservoir system, wherein the second fluid source provides a second fluid having a second predetermined analyte concentration, different from the first analyte concentration.

3. An apparatus as in claim 2, further comprising a first fluid selection system in fluid communication with the first and second fluid sources and with the second port of the flow generation and reservoir system, wherein the first fluid selection system has first and second configurations, wherein in the first configuration only the first fluid is supplied to the flow generation and reservoir system, and in the second configuration only the second fluid is supplied to the flow generation and reservoir system.

4. An apparatus as in claim 1, further comprising a plurality of additional fluid sources mounted such that each additional fluid source can be placed in fluid communication with the second port of the flow generation and reservoir system, wherein each additional fluid source provides an additional fluid having a predetermined analyte concentration, wherein the analyte concentration of the first fluid and of the additional fluids are different from each other.

5. An apparatus as in claim 4, further comprising a fluid selection system in fluid communication with the first fluid source and with each of the additional fluid sources and with the second port of the flow generation and reservoir system, and wherein the fluid control system has a plurality of configurations, wherein for the first fluid source and for each of the additional fluid sources there is a configuration of the first fluid selection system that allows only said fluid source to supply fluid to the flow generation and reservoir system.

6. An apparatus as in claim 1, further comprising a second fluid source mounted such that it can be placed in fluid communication with the second port of the analyte sensor, wherein the second fluid source provides a second fluid having a second predetermined analyte concentration, different from the first analyte concentration.

7. An apparatus as in claim 6, further comprising a first fluid selection system in fluid communication with the second port of the analyte sensor, with the first port of the flow generation and reservoir system, and with the second fluid source, wherein the fluid selection system has a first configuration in which the second port of the analyte sensor is in fluid communication with the second port of the flow generation and reservoir system and not with the second fluid source, and a second configuration in which the first port of the flow generation and reservoir system is in fluid communication with the second fluid source and not with the second port of the analyte sensor.

8. An apparatus as in claim 7, wherein the first fluid selection system comprises one or more multiway valves, or a plurality of shutoff valves, or a plurality of pinch clamps, or a combination thereof.

9. An apparatus as in claim 2, further comprising a waste channel mounted such that it can be placed in fluid communication with the first port of the analyte sensor.

10. An apparatus as in claim 9, further comprising a first fluid selection system in fluid communication with the first port of the analyte sensor, with the patient interface device, and with the waste channel, wherein the first fluid selection system has a first configuration in which the first port of the analyte sensor is in fluid communication with the patient interface device and not with the waste channel, and a second configuration in which the first port of the analyte sensor is in fluid communication with the waste channel and not with the patient interface device.

11. An apparatus as in claim 6, further comprising a waste channel mounted such that it can be placed in fluid communication with the first port of the analyte sensor.

12. An apparatus as in claim 11, further comprising a second fluid selection system in fluid communication with the first port of the analyte sensor, with the patient interface device, and with the waste channel, wherein the second fluid selection system has a first configuration in which the first port of the analyte sensor is in fluid communication with the patient interface device and not with the waste channel, and a second configuration in which the first port of the analyte sensor is in fluid communication with the waste channel and not with the patient interface device.

13. An apparatus as in claim 1, further comprising a first access port in fluid communication with the first port of the analyte sensor and allowing fluid communication with the first port of the analyte sensor, and further comprising a second access port in fluid communication with the second port of the analyte sensor allowing fluid communication with the second port of the analyte sensor.

14. An apparatus as in claim 1, further comprising a flow divider in fluid communication with the first port of the analyte sensor and with the second port of the analyte sensor.

15. An apparatus as in claim 14, wherein the fluid pathway from the patient interface device to the first port of the analyte sensor has a first flow cross-section, the fluid pathway through the analyte sensor has a second flow cross-section, and the fluid bypass has a third flow cross-section, wherein the first flow cross-section is larger than the third flow cross-section, and the third flow cross-section is larger than the second flow cross-section.

16. An apparatus as in claim 1, wherein the flow generation and reservoir system comprises a syringe pump having first and second ports, the first port in fluid communication with the second port of the analyte sensor and the second port in fluid communication with the first fluid source.

17. An apparatus as in claim 2, wherein the flow generation and reservoir system comprises a syringe pump.

18. An apparatus as in claim 6, wherein the flow generation and reservoir system comprises a syringe pump.

19. An apparatus as in claim 1, wherein the flow generation and reservoir system comprises a peristaltic pump having first and second ports, and a reservoir having first and second ports, wherein the first port of the peristaltic pump is in fluid communication with the second port of the reservoir, and wherein the first port of the reservoir is in fluid communication with the analyte sensor, and wherein the second port of the peristaltic pump is mounted such that it can be placed in fluid communication with the first fluid source.

20. An apparatus as in claim 19, wherein the reservoir comprises one or more of a bag, a flexible pillow, a syringe, a bellows device, a device that can be expanded through pressure, and an expandable fluid column.

21. An apparatus as in claim 2, wherein the flow generation and reservoir system comprises a peristaltic pump having first and second ports, and a reservoir having first and second ports, wherein the first port of the reservoir comprises the first port of the flow generation and reservoir system, the second port of the peristaltic port comprises the second port of the flow generation and reservoir system, and the first port of the peristaltic pump is in fluid communication with the second port of the reservoir.

22. An apparatus as in claim 21, wherein the reservoir comprises one or more of a bag, a flexible pillow, a syringe, a bellows device, a device that can be expanded through pressure, and an expandable fluid column.

23. An apparatus as in claim 6, wherein the flow generation and storage reservoir comprises a peristaltic pump having first and second ports, and a reservoir having first and second ports, wherein the first port of the reservoir comprises the first port of the flow generation and reservoir system, the second port of the peristaltic port comprises the second port of the flow generation and reservoir system, and the first port of the peristaltic pump is in fluid communication with the second port of the reservoir.

24. An apparatus as in claim 23, wherein the reservoir comprises one or more of a bag, a flexible pillow, a syringe, a bellows device, a device that can be expanded through pressure, and an expandable fluid column.

25. An apparatus as in claim 1, wherein the flow generation and reservoir system comprises a peristaltic pump having first and second ports, and a reservoir having a first port, wherein the first port of the peristaltic pump comprises the first port of the flow generation and reservoir system, and wherein the second port of the peristaltic pump is in fluid communication with the first port of the reservoir.

26. An apparatus as in claim 1, further comprising a second fluid source, and wherein the flow generation and reservoir system comprises a first syringe pump and a second syringe pump, wherein the first syringe pump is in fluid communication with the first fluid source, and wherein the second syringe pump is in fluid communication with the second fluid source, and wherein the first syringe pump and second syringe pump are each in fluid communication with the second port of the analyte sensor.

27. An apparatus as in claim 26, wherein the first syringe pump is connected to the second port of the analyte sensor through a first flow interrupting device, and wherein the second syringe pump is connected to the second port of the analyte sensor through a second flow interrupting device.

28. An apparatus as in claim 1, further comprising a second fluid source, and wherein the flow generation and reservoir system comprises a first reservoir and a second reservoir and a peristaltic pump having first and second ports, wherein the first reservoir is in fluid communication with the first fluid source, and wherein the second reservoir is in fluid communication with the second fluid source, and wherein the first reservoir and second reservoirs are each in fluid communication with the second port of the peristaltic pump, and wherein the first port of the peristaltic pump is in fluid communication with the second port of the analyte sensor.

29. An apparatus as in claim 28, wherein the first reservoir is connected to the second port of the peristaltic pump through a first flow interrupting device, and wherein the second reservoir is connected to the second port of the peristaltic pump through a second flow interrupting device.

30. An apparatus as in claim 1, further comprising a pressure monitor in fluid communication with the circulatory system of a patient.

31. An apparatus as in claim 2, further comprising a pressure monitor in fluid communication with the circulatory system of a patient.

32. An apparatus as in claim 6, further comprising a pressure monitor in fluid communication with the circulatory system of a patient.

33. A method of measuring an analyte concentration, comprising:
a. Providing an apparatus as in claim 1;
b. Operating the flow generation and reservoir system to place the first fluid in operative contact with the analyte sensor;
c. Determining a calibration responsive to the analyte sensor output while in operative contact with the first fluid;
d. Operating the flow generation and storage system to place blood from the patient in operative contact with the analyte sensor;
e. Determining the analyte concentration from the analyte sensor output while in operative contact with blood and from the calibration.

34. A method of measuring an analyte concentration, comprising:
a. Providing an apparatus as in claim 2;
b. Operating the flow generation and reservoir system to place the first fluid in operative contact with the analyte sensor;
c. Operating the flow generation and storage system to place the second fluid in operative contact with the analyte sensor;
d. Determining a calibration responsive to the analyte sensor output while in operative contact with the first fluid and the analyte sensor output while in operative contact with the second fluid;
e. Operating the flow generation and storage system to place blood from the patient in operative contact with the analyte sensor;
f. Determining the analyte concentration from the analyte sensor output while in operative contact with blood and from the calibration.

35. A method of measuring an analyte concentration, comprising:
a. Providing an apparatus as in claim 6;
b. Operating the flow generation and reservoir system to place the first fluid in operative contact with the analyte sensor;
c. Operating the flow generation and reservoir system to place the second fluid in operative contact with the analyte sensor;
d. Determining a calibration responsive to the analyte sensor output while in operative contact with the first fluid and the analyte sensor output while in operative contact with the second fluid;
e. Operating the flow generation and reservoir system to place blood from the patient in operative contact with the analyte sensor;
f. Determining the analyte concentration from the analyte sensor output while in operative contact with blood and from the calibration.

36. An apparatus for measuring an analyte in blood taken from a patient, comprising:
a. A patient interface device, capable of interfacing with the circulatory system of a patient;
b. An analyte sensor having first and second ports, with the first port in fluid communication with the patient interface device;
c. A flow generation and reservoir system having first and second ports, with the first port in fluid communication with second port of the analyte sensor;
d. A first fluid source, mounted such that it can be placed in fluid communication with the second port of the flow generation and reservoir system, wherein the first fluid source provides a first fluid having a first predetermined analyte concentration; and
e. A second fluid source, mounted such that it can be placed in fluid communication with the second port of the analyte sensor, wherein the second fluid source provides a second fluid having a second predetermined analyte concentration, where the second predetermined analyte concentration is different than the first predetermined analyte concentration.

37. An apparatus for the measurement of an analyte, comprising:
a. A patient interface device capable of interfacing with the circulatory system of a patient;
b. An analyte sensor having first and second ports, with the first port in fluid communication with the patient interface device;
c. A flow generation device having first and second ports, with the first port in fluid communication with second port of the analyte sensor;
d. A waste channel in fluid communication with the second port of the flow generation device through a first flow control device that allows fluid flow from the flow generation device to the waste channel but substantially prevents fluid from the waste channel to the flow generation device;
e. A first fluid source, mounted such that it can be placed in fluid communication with the second port of the flow generation device through a second flow control device that allows fluid flow from the first fluid source to the flow generation device but substantially prevents fluid from the flow generation device to the first fluid source, wherein the first fluid source provides a first fluid having a first predetermined analyte concentration.

38. An apparatus as in claim 37, further comprising a second fluid source, mounted such that it can be placed in fluid communication with the second port of the flow generation device through a third flow control device that allows fluid flow from the second fluid source to the flow generation device but substantially prevents fluid from the flow generation device to the second fluid source, wherein the second fluid source provides a first fluid having a second predetermined analyte concentration, and where the second predetermined analyte concentration is different than the first predetermined analyte concentration.

39. An apparatus as in claim 38, further comprising a first fluid selection system in fluid communication with the first and second fluid sources and with the second port of the flow generation device, wherein the first fluid selection system has first and second configurations, wherein in the first configuration only the first fluid is supplied to the flow generation device, and in the second configuration only the second fluid is supplied to the flow generation device.

40. An apparatus as in claim 37, further comprising a second fluid source mounted such that it can be placed in fluid communication with the second port of the analyte sensor, wherein the second fluid source provides a second fluid having a second predetermined analyte concentration, different from the first analyte concentration.

41. An apparatus as in claim 40, further comprising a first fluid selection system in fluid communication with the second port of the analyte sensor, with the first port of the flow generation device, and with the second fluid source, wherein the fluid selection system has a first configuration in which the second port of the analyte sensor is in fluid communication with the second port of the flow generation device and not with the second fluid source, and a second configuration in which the first port of the flow generation device is in fluid communication with the second fluid source and not with the second port of the analyte sensor.

42. A method of measuring an analyte, comprising:
a. Providing an apparatus as in claim 37;
b. Placing the patient interface device in fluid communication with the vascular system of a patient;
c. Operating the flow generation device to place the first fluid in operative contact with the analyte sensor and determining a response of the analyte sensor responsive to the first fluid;
d. Operating the flow generation device to move fluid from the analyte sensor to the waste channel and to place blood from the patient in operative contact with the analyte sensor and determining a response of the analyte sensor responsive to the blood; and
e. Determining an analyte measurement from the responses of the analyte sensor.

43. A method of measuring an analyte, comprising:
a. Providing an apparatus as in claim 38;
b. Placing the patient interface device in fluid communication with the vascular system of a patient;
c. Operating the flow generation device to place the first fluid in operative contact with the analyte sensor and determining a response of the analyte sensor responsive to the first fluid;
d. Operating the flow generation device to place the second fluid in operative contact with the analyte sensor and determining a response of the analyte sensor responsive to the second fluid;
e. Operating the flow generation device to move fluid from the analyte sensor to the waste channel and to place blood from the patient in operative contact with the analyte sensor and determining a response of the analyte sensor responsive to the blood; and
f. Determining an analyte measurement from the responses of the analyte sensor.

44. A method of measuring an analyte, comprising:
a. Providing an apparatus as in claim 40;
b. Placing the patient interface device in fluid communication with the vascular system of a patient;
c. Operating the flow generation device to place the first fluid in operative contact with the analyte sensor and determining a response of the analyte sensor responsive to the first fluid;
d. Operating the flow generation device to place the second fluid in operative contact with the analyte sensor and determining a response of the analyte sensor responsive to the second fluid;
e. Operating the flow generation device to move fluid from the analyte sensor to the waste channel and to place blood from the patient in operative contact with the analyte sensor and determining a response of the analyte sensor responsive to the blood; and
f. Determining an analyte measurement from the responses of the analyte sensor.

45. A method as in claim 34, wherein the analyte sensor is placed in operative contact with the second fluid after being in operative contact with the first fluid and before being in operative contact with blood, and wherein the analyte concentration of the second fluid is less than the analyte concentration of the first fluid.

46. A method as in claim 35, wherein the analyte sensor is placed in operative contact with the second fluid after being in operative contact with the first fluid and before being in operative contact with blood, and wherein the analyte concentration of the second fluid is less than the analyte concentration of the first fluid.

47. An apparatus as in claim 1, further comprising an access port allowing extraction of blood from the system.

48. An apparatus as in claim 1, further comprising an access port allowing extraction of blood from the system between the patient interface device and the analyte sensor.

49. An apparatus as in claim 1, further comprising an access port allowing extraction of blood from the system between the analyte sensor and the flow generation device.
